(19) United States
(12) Patent Application Publication
(10) Pub. No.: US 2023/0420076 A1
(43) Pub. Date: Dec. 28, 2023

(54) ESTIMATING HLA EXPRESSION LOSS

(71) Applicant: Genentech, Inc., South San Francisco, CA (US)

(72) Inventors: Thomas Dee WU, San Francisco, CA (US); Oliver Anthony Zill, San Francisco, CA (US); Jieming Chen, Foster City, CA (US); William Frederick Forrest, III, San Carlos, CA (US); Christian Hammer, Millbrae, CA (US)

(21) Appl. No.: 18/463,118

(22) Filed: Sep. 7, 2023

Related U.S. Application Data
(63) Continuation of application No. PCT/US2022/019409, filed on Mar. 8, 2022.
(60) Provisional application No. 63/158,306, filed on Mar. 8, 2021.

(51) Int. Cl. G16B 20/20

(52) U.S. Cl. CPC G16B 20/20

(57) ABSTRACT

A method and system for typing major histocompatibility complex (MHC) alleles. The method includes receiving a set of exon-resolution identifiers for an allele pair associated with an MHC gene. An exon-resolution identifier of the set of exon-resolution identifiers for a corresponding allele of the allele pair describes an allele group, a specific allele protein, and exon region information for the corresponding allele. A plurality of reads is received for a sample. For each exon-resolution identifier of the set of exon-resolution identifiers, a set of intron-resolution identifiers is identified to form a plurality of intron-resolution candidate identifiers. An intron-resolution identifier of the set of intron-resolution identifiers for the corresponding allele describes the allele group, the specific allele protein, the exon region information, and intron region information for the corresponding allele. A final set of intron-resolution identifiers is generated for the allele pair from the plurality of intron-resolution candidate identifiers.

### BACKGROUND

This description is generally directed towards evaluating human leukocyte antigen (HLA) loss. More specifically, this description provides methods and systems for estimating HLA loss in a subject based on alignments of reads with allele sequences identified using intron-resolution identifiers.

HLA loss is the absence or decrease of HLA allelic expression due to genetic modification, epigenetic modification, or indirect regulation. For example, HLA loss with respect to a particular HLA allele is an absence or decrease of expression of that particular HLA allele. Genetic modification is also referred to as hard modification; epigenetic modification is also referred to as soft modification. Genetic modification is irreversible, while epigenetic modification is reversible. Oftentimes, HLA loss is seen as a result of unhealthy or diseased tissue (e.g., a tumor) that evolves to evade anti-tumor immune responses. HLA loss can make it difficult to provide treatment for tumors because of the loss or low availability of a vehicle for presenting a therapeutic antigen (e.g., a neoantigen). In this manner, HLA loss can contribute, for example, to immune system evasion by cancer and to therapeutic resistance.

### SUMMARY

The embodiments described herein provide methods and systems for detecting HLA loss and/or HLA gain in a subject based on sequencing data (e.g., next generation sequencing data). The methods and systems described herein enable evaluating HLA loss and/or gain with respect to specific alleles for an HLA gene identified at the intron-region level (e.g., taking into account polymorphisms in the intron regions).

In one or more embodiments, a method is provided for typing major histocompatibility complex (MHC) alleles. The method includes receiving a set of exon-resolution identifiers for an allele pair associated with an MHC gene. An exon-resolution identifier of the set of exon-resolution identifiers for a corresponding allele of the allele pair describes an allele group, a specific allele protein, and exon region information for the corresponding allele. A plurality of reads is received for a sample. For each exon-resolution identifier of the set of exon-resolution identifiers, a set of intron-resolution identifiers is identified to form a plurality of intron-resolution candidate identifiers. An intron-resolution identifier of the set of intron-resolution identifiers for the corresponding allele describes the allele group, the specific allele protein, the exon region information, and intron region information for the corresponding allele. A final set of intron-resolution identifiers is generated for the allele pair from the plurality of intron-resolution candidate identifiers.

In one or more embodiments, a method is provided for comparing reads with allele sequences for major histocompatibility complex (MHC) genes for use in evaluating MHC allelic expression. The method includes receiving a first set of intron-resolution identifiers for a first allele for an MHC gene and a second set of intron-resolution identifiers for a second allele for the MHC gene. A first plurality of reads for a first sample and a second plurality of reads for a second sample are received. A first allele sequence for the first allele for the MHC gene is identified using a selected one of the first set of intron-resolution identifiers and a second allele sequence for the second allele for the MHC gene is identified using a selected one of the second set of intron-resolution identifiers. Allele-read alignments are quantified between the first allele sequence and each of the first plurality of reads and the second plurality of reads and between the second allele sequence and each of the first plurality of reads and the second plurality of reads to form an alignment output.

In one or more embodiments, a method is provided for analyzing major histocompatibility complex (MHC) allelic loss between samples. The method includes receiving an MHC alignment input for a pair of alleles for an MHC gene associated with a first sample and a second sample. A non-MHC alignment input is received for a plurality of non-MHC genes associated with the first sample and the second sample. An allelic loss threshold is computed using the non-MHC alignment input. A statistical analysis of the MHC alignment input is performed using the allelic loss threshold to form a statistical output for use in evaluating MHC loss.

In one or more embodiments, a method is provided for analyzing major histocompatibility complex (MHC) allelic loss between samples. The method includes receiving a first plurality of reads for a first sample and a second plurality of reads for a second sample. A first set of intron-resolution identifiers for a first allele for an MHC gene and a second set of intron-resolution identifiers for a second allele for the MHC gene are received. A set of allele sequence pairs is identified using the first set of intron-resolution identifiers and the second set of intron-resolution identifiers. Each allele sequence pair in the set of allele sequence pairs includes a first allele sequence for the first allele and a second allele sequence for the second allele. For each allele sequence pair in the set of allele sequence pairs, a plurality of alignment counts is generated using the first allele sequence, the second allele sequence, the first plurality of reads, and the second plurality of reads. An allelic loss threshold is computed using a collection of alignment counts for non-MHC genes. A statistical analysis of the plurality of alignment counts is performed using the allelic loss threshold to form a statistical output for use in evaluating MHC loss in the second sample compared to the first sample.

In one or more embodiments, a method is provided for evaluating major histocompatibility complex (MHC) copy number alteration. The method includes receiving a first plurality of reads for a first sample and a second plurality of reads for a second sample. A final set of intron-resolution identifiers is identified for an allele pair for an MHC gene using the first plurality of reads and a set covering algorithm. A set of allele sequence pairs is identified using the final set of intron-resolution identifiers. Each allele sequence pair in the set of allele sequence pairs includes a first allele sequence and a second allele sequence for the allele pair. For each allele sequence pair in the set of allele sequence pairs, a plurality of alignment counts is generated using the first allele sequence, the second allele sequence, the first plurality of reads, and the second plurality of reads. A statistical analysis of the plurality of alignment counts is performed to form a statistical output. MHC copy number alteration in the second sample is evaluated as compared to the first sample using the statistical output.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 is a schematic diagram illustrating an example of an evaluation system for evaluating HLA loss in accordance with one or more embodiments.

FIG. 2 is a flow diagram illustrating an example of a process for evaluating HLA loss in accordance with one or more embodiments.

FIG. 3 is a flow diagram illustrating an example of a process for typing HLA alleles in accordance with one or more embodiments.

FIG. 4 is a flow diagram illustrating an example of a process for generating a final set of intron-resolution identifiers in accordance with one or more embodiments.

FIG. 5 is an illustration of an example of a matrix representation of a disjunction of pairs of intron-resolution identifiers in accordance with one or more embodiments.

FIG. 6 is a flow diagram illustrating an example of a process for typing HLA alleles in accordance with one or more embodiments.

FIG. 7A is a flow diagram illustrating an example of a process for generating a set of exon-resolution identifiers in accordance with one or more embodiments.

FIG. 7B is a flow diagram illustrating an example of a process for identifying a series of a final set of exon identifiers for each exon position of an HLA gene in accordance with one or more embodiments.

FIG. 8 is a bipartite graph of allele-read alignments in accordance with one or more example embodiments.

FIG. 9 is a flow diagram illustrating an example of a process for quantifying allele-read alignments for HLA genes in accordance with one or more embodiments.

FIG. 10 illustrates allele-read alignments and alignment counts in accordance with one or more embodiments.

FIG. 11 is a flow diagram illustrating an example of a process for analyzing HLA allele expression in accordance with one or more embodiments.

FIG. 12 illustrates an example of a plot of gene variation between two samples in accordance with one or more embodiments.

FIG. 13 illustrates an example of a plot generated using a beta-binomial model in accordance with one or more embodiments.

FIG. 14 is a flow diagram illustrating an example of a process for evaluating HLA loss in accordance with one or more embodiments.

FIG. 15 illustrates an example of a Voronoi diagram in accordance with one or more embodiments.

FIG. 16 is a flow diagram illustrating an example of evaluating HLA loss in accordance with one or more embodiments.

FIGS. 17A-17F illustrate a plot series showing HLA loss detection in various tumor samples in accordance with one or more embodiments.

FIG. 18 is a block diagram illustrating an example of a computer system in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

The embodiments described herein recognize that detecting HLA loss and/or gain (e.g., via HLA copy number alteration) is important to developing and/or managing certain types of immunotherapies such as, for example, but not limited to, cancer immunotherapy. For example, a tumor can use various mechanisms (e.g., genetic modification, epigenetic modification, or indirect regulation) to cause HLA loss that enables the tumor to escape or evade therapy and have a selective advantage. For example, a particular immunotherapy (e.g., a T cell therapy) may be ineffective and unable to recognize a tumor-specific antigen (e.g., a neoantigen) and thereby activate when there is HLA loss. For example, a tumor cell that does not have or that has a reduced expression of a particular HLA allele or HLA alleles may not be recognized or killed by T cells that are reactive to a given antigen on that tumor cell. Thus, detecting HLA loss in a subject's tumor, for example, can help develop and/or personalize immunotherapies such as, but not limited to, T-cell therapies and/or natural killer (NK) cell therapies.

The embodiments described herein provide various methods, systems, and non-transitory computer readable media for evaluating HLA loss in a sample obtained from a subject as compared to another sample. For example, the embodiments described herein enable analyzing HLA allele expression in two samples to estimate whether there is any HLA expression loss between the two samples (e.g., reduced or absent expression of an HLA allele in one sample as compared to expression of that HLA allele in the other sample). The embodiments described herein may be used to characterize HLA expression between two samples. In various examples, these two samples include a normal or otherwise healthy sample and a tumor or otherwise unhealthy or diseased sample.

In one or more example embodiments, HLA alleles are typed at an intron level of resolution. For example, HLA alleles can be typed based on variances (e.g., polymorphisms) within the exon regions of these HLA alleles and based on variances (e.g., polymorphisms) within the intron regions of these HLA alleles. Using such an intron-resolution identifier for an HLA allele allows the allele sequence for that HLA to be more accurately identified which may enable improved alignments between HLA alleles and reads obtained from sequencing.

Recognizing that some currently available methods for typing HLA alleles mainly provide exon level of resolution (without or with only partial intron region information), the embodiments described herein enable typing HLA alleles at the intron level of resolution to improve overall accuracy in detecting HLA loss. For example, HLA alleles are first typed with exon-resolution identifiers to narrow the field of possible alleles that need to be compared to sequencing data. With this narrowed field, intron-resolution identifiers corresponding to these exon-resolution identifiers can be compared to sequencing data (e.g., reads) as compared to having to compare all possible intron-resolution identifiers to the sequencing data. The typing of the HLA alleles using the exon-resolution identifiers can be performed more accurately by first narrowing the field of possible exon-resolution identifiers to those having exons that match to the reads. Further, the typing of the HLA alleles using the exon-resolution identifiers can be performed more efficiently, faster, and/or using fewer computing resources by first narrowing the field of possible exon-resolution identifiers to those having exons that match to the reads. By first narrowing at the individual exon level, computational time and resource savings can be realized.

The embodiments described herein may be used in various ways to develop and/or personalize immunotherapy such as T cell therapies or cancer vaccines. For example, if HLA loss is estimated as being present in a tumor sample (e.g., a sample of tumor tissue) for one HLA allele, a T cell therapy or cancer vaccine may be designed to be reactive to an antigen presented by another HLA allele in that patient for which HLA loss is not estimated or detected. This type of HLA loss detection may be crucial to providing effective and timely therapy to individual subjects.

### II. Definitions

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology, and toxicology as described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

Where reference is made to a list of elements (e.g., elements a, b, c), such reference is intended to include any one of the listed elements by itself, any combination of less than all of the listed elements, and/or a combination of all of the listed elements.

As used herein, a “model” includes at least one of an algorithm, a formula, a mathematical technique, a machine algorithm, a probability distribution or model, or another type of mathematical or statistical representation.

As used herein, a “subject” may refer to a mammal being assessed for treatment and/or being treated, a mammal participating in a clinical trial, a mammal undergoing anti-cancer therapies, or any other mammal of interest. In various embodiments, the terms “subject,” “individual,” and “patient” are used interchangeably herein. A subject can be a healthy or asymptomatic individual, an individual that has or is suspected of having a disease (e.g., cancer) or a pre-disposition to the disease, an individual that is in need of therapy or suspected of needing therapy, or a combination thereof. A subject may be, for example, without limitation, an individual having cancer or an individual having an autoimmune disease. A subject may be human. In other cases, a subject may be some other type of mammal. For example, a subject may be a mammal used in forming laboratory models for human disease. Such mammals include, but are not limited to, mice, rats, primates (e.g., cynomolgus monkey), etc.

As used herein, a “sample” can refer to “biological sample” of a subject. A sample can include tissue (e.g., a biopsy), single cell, multiple cells, fragments of cells or an aliquot of body fluid. The sample may have taken from a subject, by means including venipuncture, excretion, ejaculation, massage, biopsy, needle aspirate, lavage sample, scraping, surgical incision, or intervention or other means known in the art.

As used herein, a “nucleotide,” comprises a nucleoside and a phosphate group. A “nucleoside,” as used herein, comprises a nucleobase and a five-carbon sugar (e.g., ribose, deoxyribose, or analogs thereof). When the nucleobase is bonded to ribose, the nucleoside may be referred to as a ribonucleoside. When the nucleobase is bonded to deoxyribose, the nucleoside may be referred to as a deoxyribonucleoside. A “nucleobase,” which may be also referred to as a “nitrogenous base,” can take the form of one of five types: adenine (A), guanine (G), thymine (T), uracil (U), and cytosine (C).

As used herein, a “polynucleotide,” “nucleic acid,” or “oligonucleotide” refers to a linear polymer of nucleotides (or nucleosides joined by internucleosidic linkages). Generally, a polynucleotide comprises at least three nucleotides. Generally, an oligonucleotide is comprised of nucleotides that range in number from a few nucleotides (or monomeric units) to several hundreds of nucleotides (monomeric units). Whenever a polynucleotide such as an oligonucleotide is represented by a sequence of letters, such as “ATGCCTG,” it will be understood that the nucleotides are in 5′-3′ order or direction from left to right and that “A” denotes adenine, “C” cytosine, “G” denotes guanine, and “T” denotes thymine, unless otherwise noted. The letters A, C, G, and T may be used to refer to the nucleobases themselves, as described above, the nucleosides that include those nucleobases, or the nucleotides that include those bases, as is standard in the art.

Deoxyribonucleic acid (DNA) is a chain of nucleotides consisting of 4 types of nucleotides: adenine (A), thymine (T), cytosine (C), and guanine (G). Ribonucleic acid (RNA) is comprised of 4 types of nucleotides: A, C, G, and uracil (U). Certain pairs of nucleotides specifically bind to one another in a complementary fashion, which may be referred to as complementary base pairing. For example, C pairs with G and A pairs with T. In the case of RNA, however, A pairs with U. When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” “fragment sequence,” or “nucleic acid sequencing read” denotes any information or data that is indicative of the order of the nucleotide bases (e.g., A, C, G, T/U) in a molecule (e.g., whole genome, whole transcriptome, exome, oligonucleotide, polynucleotide, fragment, etc.) of DNA or RNA. It should be understood that the present disclosure contemplates that this sequence information may be obtained using any of the available varieties of techniques, platforms, or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic-based systems, etc., or a combination thereof.

A term “genome,” as used herein, refers to the genetic material of a cell or organism, including animals, such as mammals (e.g., humans), and comprises nucleic acids, such as DNA. A genome is stored on one or more chromosomes comprised of DNA sequences. In humans, DNA includes, for example, genes, noncoding DNA, and mitochondrial DNA. The human genome typically contains 23 pairs of chromosomes: 22 pairs of autosomal chromosomes (autosomes) plus the sex-determining X and Y chromosomes. The 23 pairs of chromosomes include one copy from each parent. The DNA that makes up the chromosomes is referred to as chromosomal DNA and is present in the nucleus of human cells (nuclear DNA).

As used herein, a “gene” is a discrete portion of heritable, genomic sequence which affect a subject's traits by being expressed as a functional product or by regulation of gene expression. The total complement of genes in a subject or cell is known as the subject's or cell's genome. A region of a chromosome at which a particular gene is located is called its locus. Each locus contains one allele of a gene. Thus, a pair of chromosomes together has two loci that each contain an allele of the gene to form an allele pair. The two alleles may be the same or may be different (e.g., have slightly varying gene sequences).

As used herein, an “allele” is a variant of a gene. One allele of a gene may differ from another allele of the same gene in various ways. For example, two alleles for a same gene may differ by, for example, a protein (e.g., differences within the amino acid sequence of the encoded protein), other (silent or synonymous) variances in the exon regions that do not affect the amino acid sequence, variances in the intron regions, or some combination of these variances.

As used herein, a “sequence” denotes any information or data that is indicative of the order of the nucleotide bases (e.g., A, C, G, T/U) in a molecule (e.g., whole genome, whole transcriptome, exome, oligonucleotide, polynucleotide, fragment, etc.) of DNA or RNA. Sequence information may be obtained using any of the available varieties of techniques, platforms, or technologies, including, but not limited to, capillary electrophoresis, microarrays, ligation-based systems, polymerase-based systems, hybridization-based systems, direct or indirect nucleotide identification systems, pyrosequencing, ion- or pH-based detection systems, electronic-based systems, etc., or a combination thereof. As one example, sequence information may be obtained using next generation sequencing.

As used herein, “next generation sequencing” (NGS) refers to sequencing technologies having increased throughput as compared to traditional Sanger- and capillary electrophoresis-based approaches. These sequencing technologies have, for example, the ability to generate hundreds of thousands of relatively small sequence reads or “reads” at a time. Some examples of next generation sequencing techniques include, but are not limited to, sequencing by synthesis, sequencing by ligation, and sequencing by hybridization.

As used herein, a “read” or “sequence read” includes a string of nucleic acid bases corresponding to a nucleic acid molecule that has been sequenced. For example, a read can refer to the sequence of nucleotides determined for a nucleic acid fragment that has been subjected to sequencing, such as, for example, next generation sequencing (“NGS”). Reads can be any sequence of any number of nucleotides, with the number of nucleotides defining the read length.

As used herein, a “major histocompatibility complex gene” or “MHC gene” is a gene that encodes a system, complex, or group of cell-surface proteins responsible for the regulation of the immune system.

As used herein, a “human leukocyte antigen gene” or “HLA gene” is a gene that encodes a system, complex, or group of cell-surface proteins responsible for the regulation of the immune system. An HLA system or complex is encoded by the MHC gene complex in humans. MHC molecules that present antigens on cells are categorized as belonging to one of three classes of MHC molecules, MHC class I, MHC class II, and MHC class III. Certain HLA genes including, for example, HLA-A, HLA-B, HLA-C, correspond to MHC class I. Certain HLA genes including, for example, HLA-DP, HLA-DM, HLA-DO, HLA-DQ, and HLADR, correspond to MHC class II. HLA genes that are known include, for example, HLA-A, HLA-B, HLA-C, HLA-E, HLA-F, HLA-G, HLA-H, HLA-J, HLA-K, HLA-L, HLA-N, HLA-P, HLA-S, HLA-T, HLA-U, HLA-V, HLA-W, HLA-X, HLA-Y, HLA-Z, HLA-DRA, HLA-DRB, HLA-DQ, HLA-DOA, HLA-DOB, HLA-DMA, HLA-DMB, HLA-DPA, HLA-DPB, and HFE. Other genes that are found in the HLA region include, for example, TAP1, TAP2, PSMB9, PSMB8, MICA, MICB, MICC, MICD, and MICE.

As used herein, a “T cell”, also known as a T lymphocyte, refers to a type of an adaptive immune cell. T cells develop in the thymus gland and play a central role in the immune response of the body. T cells can be distinguished from other lymphocytes by the presence of a T cell receptor (TCR) on the cell surface. These immune cells originate as precursor cells, derived from bone marrow, and then develop into several distinct types of T cells once they have migrated to the thymus gland. T cell differentiation continues even after they have left the thymus. T cells include, but are not limited to, helper T cells, cytotoxic T cells, memory T cells, regulatory T cells, and killer T cells. Helper T cells stimulate B cells to make antibodies and help killer cells develop. Based on the T cell receptor chain, T cells can also include T cells that express αβ TCR chains, T cells that express γδ TCR chains, as well as unique TCR co-expressors (i.e., hybrid αβ-γδ T cells) that co-express the αβ and γδ TCR chains.

T cells can also include engineered T cells that can attack specific cancer cells. Engineered T cells may be designed to recognize MHC-presented peptides. For example, an engineered T cell may be designed with an antigen that is not subject to HLA loss. Engineered T cells can be formed by the millions or billions in the laboratory and then infused into a patient's body. Engineered T cells may be designed to multiply and recognize the cancer cells that express a specific protein or neoantigen. This type of technology may be used in potential next-generation immunotherapy treatment.

As used herein, “immunotherapy” refers to a treatment or class of treatments that uses one or more parts of a subject's immune system to fight a disease such as, for example, without limitation, cancer. Immunotherapy can use substances made by the body or synthesized outside of the body to improve how the immune system works to find and destroy cancer cells.

As used herein, a “neoantigen” is a tumor-specific antigen derived from somatic mutations in tumors and presented by a subject's cancer cells and antigen presenting cells. Neoantigen therapies, such as, but not limited to, neoantigen vaccines, are a relatively new approach for providing individualized cancer treatment. Neoantigen vaccines can prime a subject's T cells to recognize and attack cancer cells expressing one or more particular tumor neoantigens. This approach generates a tumor-specific immune response that spares healthy cells while targeting tumor cells. The individualized vaccine may be engineered or selected based on a subject-specific tumor profile. The tumor profile can be defined by determining DNA and/or RNA sequences from a subject's tumor cell and using the sequences to identify neoantigens that are present in tumor cells but absent in normal cells.

### III. Evaluation of HLA Loss

The embodiments described herein are generally presented with respect to HLA loss (e.g., HLA allelic expression loss) for a given HLA gene. It should be understood, however, that the embodiments described herein would be similarly used to evaluate HLA loss for multiple HLA genes. For example, various processes described below for evaluating HLA allele loss for a given HLA gene may be repeated or modified as needed to enable evaluating HLA allele loss across multiple HLA genes.

Further, the embodiments described herein are generally presented with respect to HLA loss (e.g., HLA allelic expression loss) for a given HLA gene. It should be understood, however, that the embodiments described herein would be similarly used to evaluate MHC loss for one or more MHC genes. Accordingly, terms used herein that include the modifier “HLA” (e.g., HLA loss, HLA alignment input, HLA allele, HLA gene, etc.) may be interchangeable with the modifier MHC (e.g., MHC loss, MHC alignment input, MHC allele, MHC gene, etc.).

Still further, the embodiments described herein are generally presented with respect to HLA loss (e.g., HLA allelic expression loss). It should be understood, however, that the embodiments described herein would be similarly used to evaluate HLA gain for one or more HLA genes. Thus, the various methods and systems described below for evaluating HLA allele loss for a given HLA gene may be repeated or modified as needed to enable evaluating HLA allele gain across one or more HLA genes. Further, it should be understood that in various embodiments, references to HLA loss may be interchangeable with one or more other forms of HLA copy number alteration, which is an alteration in the number of copies of at least one HLA allele for an HLA gene. In the embodiments described herein, HLA copy number alteration includes copy-neutral loss of heterozygosity (LOH), in which a first allele for a gene is lost (resulting in zero copies of the first allele) and a second allele for that gene is gained (resulting in two copies of the second allele).

### III.A System for Evaluating HLA Loss

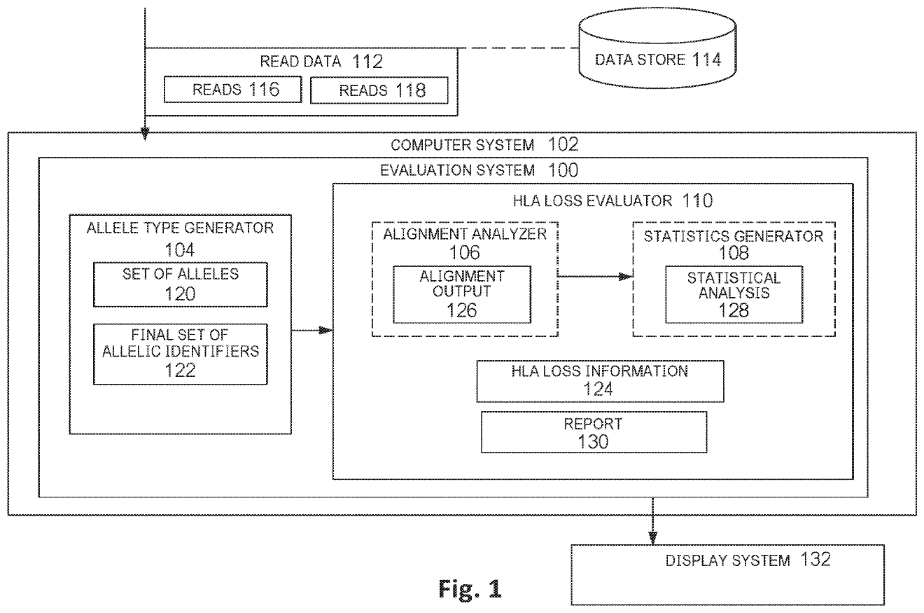

FIG. 1 is a schematic diagram illustrating an example of an evaluation system 100 for evaluating HLA loss in accordance with one or more embodiments. Evaluation system 100 is implemented using hardware, software, firmware, or a combination thereof. Evaluation system 100 may be implemented using, for example, computer system 102. Computer system 102 includes a single computer or multiple computers in communication with each other. When computer system 102 includes multiple computers, in some embodiments, one computer may be located remotely with respect to at least one other computer.

Evaluation system 100 includes allelic type generator 104, alignment analyzer 106, statistics generator 108, or a combination thereof. Each of allelic type generator 104, alignment analyzer 106, and statistics generator 108 is implemented using hardware, software, firmware, or a combination thereof. For example, each of allelic type generator 104, alignment analyzer 106, and statistics generator 108 can be implemented as a distinct compiled computer program, interpreted language script, another type of software, or a combination thereof. Alignment analyzer 106 and statistics generator 108 form HLA loss evaluator 110. HLA loss evaluator 110 can be implemented in various ways.

In one or more embodiments, alignment analyzer 106 and statistics generator 108 are separate programs with alignment analyzer 106 generating an output that is sent as input into statistics generator 108. In other embodiments, alignment analyzer 106 and statistics generator 108 are integrated or the actions that would be performed by alignment analyzer 106 and statistics generator 108 are integrated to form HLA loss evaluator 110. Accordingly, HLA loss evaluator 110 is implemented using hardware, software, or a combination thereof. In some embodiments, HLA loss evaluator 110 is implemented a compiled computer program, interpreted language script, another type of software, or a combination thereof. In other embodiments, HLA loss evaluator 110 is implemented as a plurality of programs working together. Reference herein to HLA loss evaluator 110 refers to alignment analyzer 106, statistics generator 108, a combination of alignment analyzer 106 and statistics generator 108, operations that would be performed by alignment analyzer 106, operations that would be performed by statistics generator 108, operations that would be performed by a combination of alignment analyzer 106 and statistics generator 108, or a combination thereof.

Evaluation system 100 receives read data 112 as input. One or more of allelic type generator 104 and HLA loss evaluator 110 (e.g., alignment analyzer 106, statistics generator 108, or both) receives read data 112 as input. Read data 112 includes one or more datasets. Read data 112 includes, for example, one or more sequencing datasets. A sequencing dataset includes, for example, a plurality of reads. In some embodiments, evaluation system 100 retrieves read data 112 from data store 114. Data store 114 includes, for example, but is not limited to, at least one of a database, a data storage unit, a spreadsheet, a file, a server, a cloud storage unit, a cloud database, or some other type of data store. In some examples, data store 114 comprises one or more data storage devices separate from but in communication with computer system 102. In other examples, data store 114 is at least partially integrated as part of computer system 102.

Read data 112 includes reads (e.g., sequence reads) that are generated using, for example, one or more next-generation sequencing (NGS) systems. The reads are generated using, for example, whole-exome sequencing (WES), whole genome sequencing (WGS), or both. The reads can be generated using, for example, paired-end sequencing.

For example, read data 112 includes at least a plurality of reads 116. Reads 116 are generated for a corresponding first sample which may be, for example, a biological sample. The first sample can be obtained from, for example, a subject (e.g., a live subject). In some embodiments, read data 112 further includes a plurality of reads 118 that are generated for a corresponding second sample that is different from the first sample. This second sample may be, for example, a biological sample obtained from a subject that is the same as or different from the subject from which reads 116 are generated. Reads 118 are, in some examples, generated via simulation, via a sampling from a collection of reads generated for multiple subjects, or in some other manner.

In one or more embodiments, reads 116 and reads 118 are paired-end reads. For example, paired-end sequencing of a fragment results in two sequences, a sequence generated beginning at the 5′ end of the fragment, and a sequence generated beginning at the 3′ end of the fragment. These two sequences form a paired-end read.

A biological sample for which at least a portion (e.g., reads 116, reads 118) of read data 112 is generated may be, for example, a sample of unhealthy or diseased tissue, a sample of tumor tissue, a sample of tissue that includes tumor cells, a sample of healthy or normal tissue, a sample of tissue that includes normal cells, a sample of tissue taken at a first stage or point in time during a cancer progression, a sample of tissue taken at a second stage or point in time during the cancer progression, or another type of sample. In one or more embodiments, reads 116 are generated for a sample of healthy or normal tissue and reads 118 are generated for a sample of unhealthy or diseased tissue (e.g., a tumor). In some examples, reads 116 are referred to as normal reads or healthy reads, and reads 118 are referred to as unhealthy reads, diseased reads, or tumor reads.

Allelic type generator 104 generates at least one applicable or probable allelic type for one or more genes of interest within a sample using read data 112 (e.g., based on reads 116 or reads 118). Allelic type generator 104 identifies, in one or more embodiments, a set of alleles 120 relevant to a subject using reads 116 generated for the subject. Set of alleles 120 is a set of HLA alleles that is determined to most likely be present within the sample from which reads 116 are generated for a given HLA gene. Allelic type generator 104 identifies a set of alleles 120 by identifying a final set of allelic identifiers 122 for set of alleles 120. An allelic identifier for an allele can take various forms. For example, an allelic identifier may be comprised of various letter and/or digits that form one or more fields for representing different pieces of information about an allele. An allelic identifier can have varying levels of resolution in which higher levels of resolution provide more information than lower levels of resolution. In some cases, additional letters and/or digits provide additional information. As one example, a 6-digit allelic identifier (or 6-digit identifier) has a lower resolution than an 8-digit allelic identifier (or 8-digit identifier). In some embodiments, allelic identifiers are referred to by the number of fields of information represented in these allelic identifiers. For example, a 6-digit identifier may be referred to as or generally provide the same level of information as a 3-field identifier. An 8-digit identifier may be referred to as or generally provide the same level of information as a 4-field identifier.

An HLA allele for an HLA gene can be identified using identifiers of varying resolutions including, but not limited to, exon-resolution identifiers and intron-resolution identifiers. An exon-resolution identifier for an HLA allele describes an allele group, a specific allele protein, and exon region information for the corresponding HLA allele. In one or more embodiments, the exon-resolution identifier is a 6-digit identifier in which the first and second digits identify the allele group; the third and fourth digits identify the specific allele protein; and the fifth and sixth digits identify the exon region information. The specific allele protein is determined based on DNA sequence and differences within the amino acid sequence of the encoded protein. The exon region information captures changes in one or more exon regions of the HLA allele such as, for example, synonymous nucleotide substitutions. The 6-digit identifier includes one or more letters that indicate the corresponding HLA gene, a level of expression, or both. In other embodiments, the exon-resolution identifier is a 3-field identifier in which each field is comprised of any number of letters, digits, symbols, or combination thereof.

An intron-resolution identifier provides more information than an exon-resolution identifier and therefore has a higher level of resolution than an exon-resolution identifier. An intron-resolution identifier for an HLA allele describes an allele group, a specific allele protein, exon region information, and intron region information for the corresponding HLA allele. In one or more embodiments, the intron-resolution identifier is an 8-digit identifier that adds, to a 6-digit identifier as described above, seventh and eighth digits that identify intron region information. The intron region information captures changes in one or more intron regions of the HLA allele such as, for example, polymorphisms in the intron regions. Final set of allelic identifiers 122 is a final set of intron-resolution identifiers in one or more embodiments. The 8-digit identifier includes one or more letters that indicate the corresponding HLA gene, a level of expression, or both. In other embodiments, the intron-resolution identifier is a 4-field identifier that adds one field (e.g., comprised of any number of letters, digits, symbols, or combination thereof) to the 3-field identifier.

Allele type generator 104 outputs final set of allelic identifiers 122 that correspond to a given HLA gene as determined using reads 116. HLA loss evaluator 110 receives final set of allelic identifiers 122 as input. HLA loss evaluator 110 uses final set of allelic identifiers 122 and read data 112 to generate HLA loss information 124. HLA loss information 124 may include various pieces of information that describe HLA loss between the two samples from reads 116 and reads 118. For example, HLA loss information 124 includes one or more pieces of information that can be used to quantify and/or qualify HLA loss in the sample associated with reads 118 as compared to the sample associated with reads 116.

As previously described in Section II above, HLA loss of an HLA allele for an HLA gene refers to an absence or decrease of expression of that HLA allele. HLA loss information 124 provides information that can be used to quantify and/or qualify this absence or decrease of expression. An example of information included in HLA loss information 124 is statistics generated based on alignment between an allelic identifier for an HLA allele and various reads. Another example of information included in HLA loss information 124 is one or more conclusions or inferences made using statistics. Yet another example of information included in HLA loss information 124 is an estimation of the amount of HLA loss (e.g., a percentage, etc.).

In one or more embodiments, HLA loss information 124 is generated by operations involving alignment analyzer 106 and statistics generator 108. Alignment analyzer 106, for example, receives final set of allelic identifiers 122 as input and generates alignment output 126 based on this input. Alignment analyzer 106 performs an analysis of the alignment of each allele identified by a corresponding one of final set of allelic identifiers 122 with reads 116 and reads 118. Alignment output 126 provides a quantification of these alignments. In one or more embodiments, alignment output 126 also includes a quantification of alignments between non-HLA genes with reads 116 and reads 118.

Alignment analyzer 106 outputs alignment output 126 and statistics generator 108 receives alignment output 126 as input. Statistics generator 108 performs statistical analysis 128 using alignment output 126 and, in some cases, other information. Statistical generator 108 performs statistical analysis 128 using one or more algorithms for generating statistics, one or more mathematical formulas or equations, one or more other types of analysis techniques, or a combination thereof. HLA loss evaluator 110 generates HLA loss information 124 using the results of statistical analysis 128, one or more inferences or conclusions made based on the results of statistical analysis 128, or a combination thereof.

HLA loss evaluator 110 generates, in some embodiments, report 130 using HLA loss information 124. Report 130 may include, for example, without limitation, at least one of a table, a spreadsheet, a database, a file, a presentation, an alert, a graph, a chart, one or more graphics, or a combination thereof. HLA loss evaluator 110 optionally displays report on display system 132. Display system 132 comprises one or more display devices in communication with computer system 102. Display system 132 may be separate from or at least partially integrated as part of computer system 102.

Although the operations of evaluation system 100 are generally described with respect to a given HLA gene, evaluation system 100 is capable of identifying a set of alleles for each of a plurality of HLA genes in a sample and evaluating HLA loss using the identified sets of alleles. Further, although allelic type generator 104 is shown as part of evaluation system 100, in other embodiments, allelic type generator 104 may be separate from evaluation system 100.

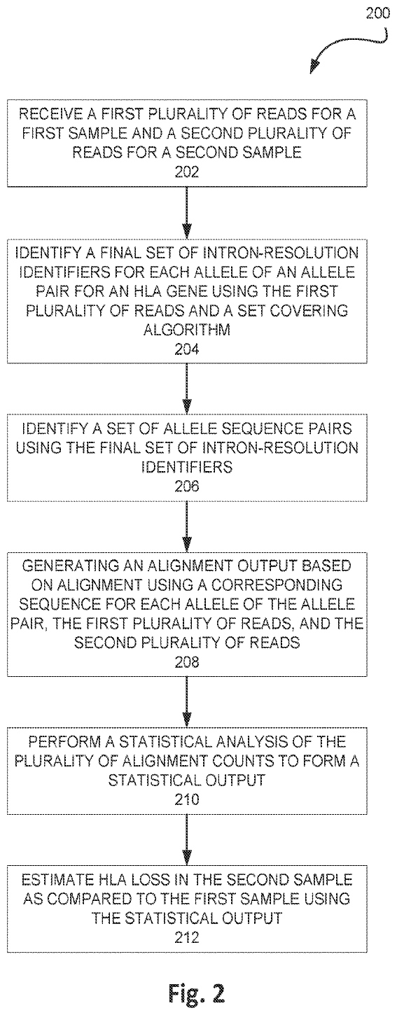

FIG. 2 is a flow diagram illustrating an example of a process 200 for evaluating HLA loss in accordance with one or more embodiments. Process 200 is one example of a process that is implemented by evaluation system 100 or at least a portion of evaluation system 100 in FIG. 1.

Step 202 includes receiving a first plurality of reads for a first sample and a second plurality of reads for a second sample. The first sample and the second sample are biological samples. The first sample may be, for example, but is not limited to, a sample of healthy or normal tissue. The second sample may be, for example, but is not limited to, a sample of unhealthy or diseased tissue (e.g., a tumor). In one or more embodiments, the first plurality of reads includes reads 116 in FIG. 1 and the second plurality of reads includes reads 118 in FIG. 1.

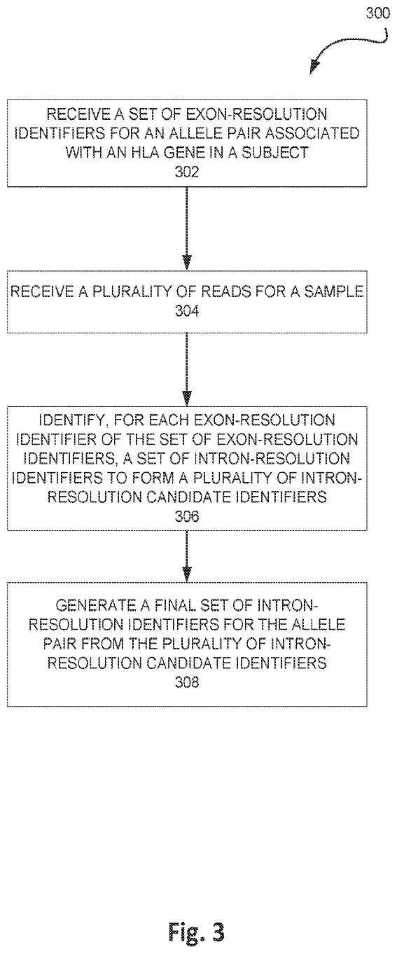

Step 204 includes identifying a final set of intron-resolution identifiers for each allele of an allele pair for an HLA gene using the first plurality of reads and a set covering algorithm. When the two alleles of the allele pair are the same, the final set of intron-resolution identifiers is the same for each allele of the allele pair. Alternatively, the allele pair may be a heterozygous allele pair such that the final set of intron-resolution identifiers is different for each allele of the allele pair. Examples of processes that may be used to implement step 204 are described in further detail in FIGS. 3-7 below in Section III.B.

Step 206 includes identifying a set of allele sequence pairs using the final set of intron-resolution identifiers. Each allele sequence pair in the set of allele sequence pairs includes a first allele sequence and a second allele sequence for the allele pair. In some examples, a single allele sequence pair is formed. In other examples, multiple unique allele sequence pairs are formed from the final set of intron-resolution identifiers. When the final set of intron-resolution identifiers includes a single intron-resolution identifier, an allele sequence corresponding to the intron-resolution identifier is used twice (e.g., for homozygosity) to form an allele sequence pair.

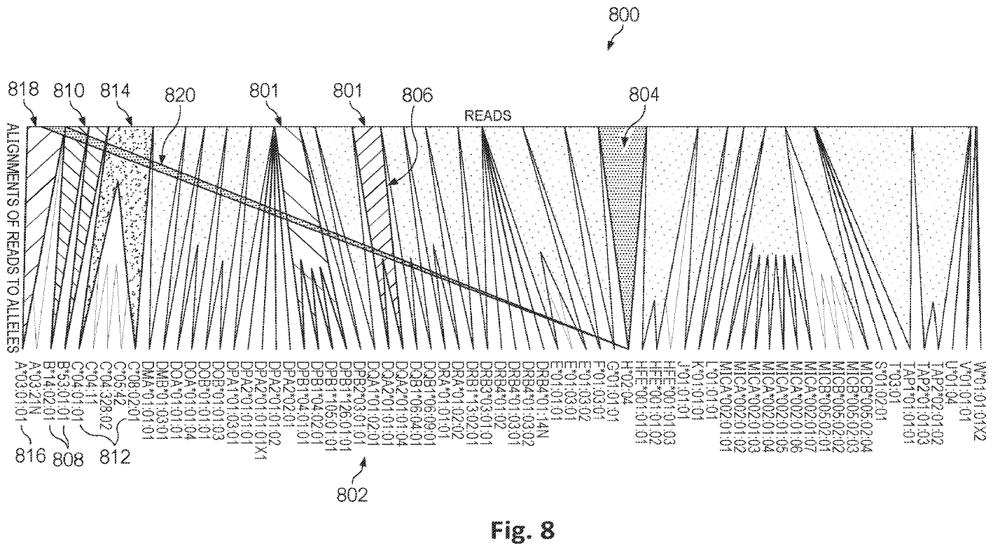

Step 208 includes generating, for each allele sequence pair in the set of allele sequence pairs, a plurality of alignment counts using the first allele sequence, the second allele sequence, the first plurality of reads, and the second plurality of reads. An example of a process used to implement step 208 is described in further detail in FIG. 8 below in Section III.C.1. The plurality of alignment counts generated in step 208 may be one example of information included in an alignment output (e.g., alignment output 126 in FIG. 1). Thus, step 208 may be one example of a manner for generating an alignment output using the first plurality of reads, the second plurality of reads, and the set of allele sequence pairs.

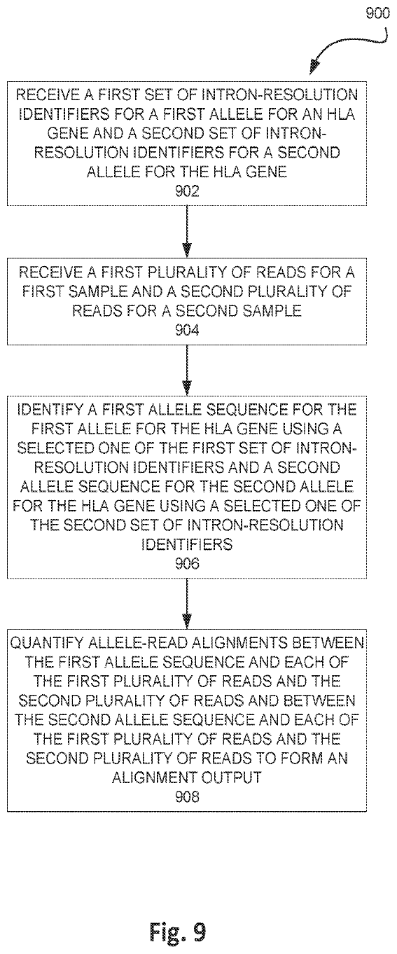

Step 210 includes performing a statistical analysis for the set of allele sequence pairs based on the alignment output. The statistical analysis is performed to generate a statistical output that is used to evaluate HLA loss. An example of a process used to implement step 210 is described in further detail in FIG. 9 below in Section III.C.2. Step 210 includes, for example, providing information about the expression of the alleles in the allele pair for the allele gene. In one or more embodiments, step 210 includes providing at least a portion of this information with respect to an overall gene variation described by or otherwise inferred from alignments between non-HLA alleles and each of the first plurality of reads and the second plurality of reads.

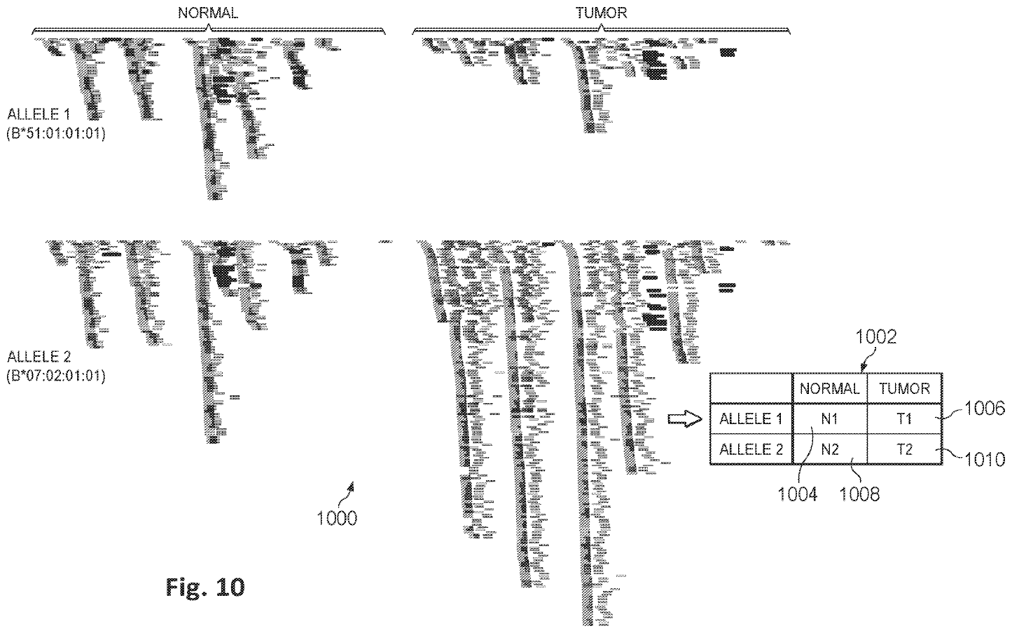

Step 212 includes evaluating HLA loss based on the statistical analysis performed. Step 212 includes, for example, generating inferences or conclusions based on the statistical analysis performed in step 212. In one or more embodiments, step 212 includes determining whether expression of the alleles in the allele pair is normal or indicates a single deletion, double deletion, loss of heterozygosity (LOH) such as, for example, copy-neutral LOH, single amplification, double amplification, or another genetic/chromosomal event/mutation. Thus, step 212 can include estimating whether HLA loss is present. An example of a process used to implement step 212 is described in further detail in FIG. 10 below in Section III.C.3.

The evaluation performed in step 212 may be used to make various decisions regarding the subject. These decisions may be made with respect to, for example, diagnosis, treatment, prediction of disease progression/outcome, or a combination thereof. Examples of some types of decisions that may be made are described below in Section III.D.

III.B Typing of HLA Alleles

Before HLA loss in a sample of a subject is evaluated, the HLA alleles present in the subject are identified. The subject is HLA-typed. This HLA typing can be performed with respect to each known HLA gene (e.g., HLA-A, HLA-B, HLA-C, etc.). In some cases, the exact two HLA alleles present in a subject for each HLA gene in the subject are identified. These two HLA alleles may be the same (i.e., homozygosity) or different (i.e., heterozygosity). In other cases, the most likely options for the two HLA alleles present in the subject for each HLA gene are identified.

III.B.1 HLA Typing to Identify Intron-Resolution Identifiers

FIG. 3 is a flow diagram illustrating an example of a process 300 for typing HLA alleles in accordance with one or more embodiments. Process 300 is one example of a process that is implemented by evaluation system 100 or at least a portion of evaluation system 100 in FIG. 1. Process 300 may be implemented by, for example, allelic type generator 104 in FIG. 1. Process 300 is one example of a process that may be included in step 204 in FIG. 2 described above.

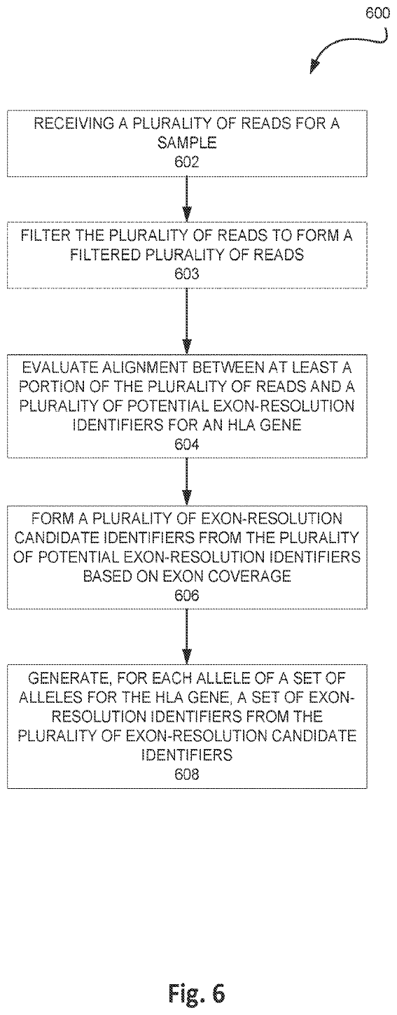

Step 302 includes receiving a set of exon-resolution identifiers for each allele of a set of alleles associated with an HLA gene in a subject. As previously described, an exon-resolution identifier, such as one of the set of exon-resolution identifiers for a corresponding allele of the set of alleles, describes an allele group, a specific allele protein, and exon region information for the corresponding allele. In one or more embodiments, the set of exon-resolution identifiers is received as input from a different system (e.g., input received from an HLA typing from High-quality Dictionary system (HLA-HD)). In other embodiments, the set of exon-resolution identifiers is an example of an output of, for example, allelic type generator 104 in FIG. 1. One example of a manner in which the set of exon-resolution identifiers can be identified is described in FIG. 6 below.

In step 302, a set of exon-resolution identifiers may be received for a single allele associated with an HLA gene in the case of homozygosity at the exon level. For example, because the set of exon-resolution identifiers would be identical for the allele pair associated with the HLA gene, a single allele (and therefore a single set of exon-resolution identifiers) is used in step 302. This type of narrowing or filtering reduces the overall time and/or processing resources associated with performing process 300.

Step 304 includes receiving a plurality of reads for a sample. The sample may be, for example, a biological sample obtained from the subject. The sample may be, for example, of healthy or normal tissue. The sample may be, for example, of unhealthy or diseased tissue taken at a point in time that is of interest. The plurality of reads include, in some embodiments, reads generated via WES or WGS. In other embodiments, the plurality of reads include reads generated via simulation, via a sampling from a collection of reads generated for multiple subjects, or via some other methodology.

Step 306 includes identifying, for each exon-resolution identifier of the set of exon-resolution identifiers for each allele of the set of alleles, a set of intron-resolution identifiers to form a plurality of intron-resolution candidate identifiers. As previously described, an intron-resolution identifier, such as one of the set of intron-resolution identifiers for the corresponding allele, describes the allele group, the specific allele protein, the exon region information, and intron region information for the corresponding allele. Thus, the intron-resolution identifier for a given HLA allele provides a higher level of resolution than the exon-resolution identifier for the same HLA allele.

In various embodiments, step 306 includes retrieving from a data store, such as data store 114 in FIG. 1, or another data source (e.g., the international ImMunoGeneTics information System® (IMGT®) database), known intron-resolution identifiers (e.g., 8-digit identifiers) for a given exon-resolution identifier (e.g., 6-digit identifier). For example, a particular HLA gene (e.g., HLA-A, HLA-B, HLA-C, etc.) may have P known combinations of polymorphisms in the intron regions. Thus, the intron region information may identify P different possible intron-resolution candidate identifiers for a given exon-resolution identifier for that HLA gene.

Step 308 includes generating, for each allele of the set of alleles, a final set of intron-resolution identifiers from the plurality of intron-resolution candidate identifiers. The final set of intron-resolution identifiers including a single intron-resolution identifier indicates homozygosity at the intron-resolution level. The final set of intron-resolution identifiers including two intron-resolution identifiers, one for each of the two HLA alleles expected for the HLA gene, indicates heterozygosity at the intron-resolution level. In some embodiments, the final set of intron-resolution identifiers includes multiple intron-resolution identifiers for at least one of the two alleles. Thus, the final set of intron-resolution identifiers may be a pair of disjunctive intron-resolution identifiers.

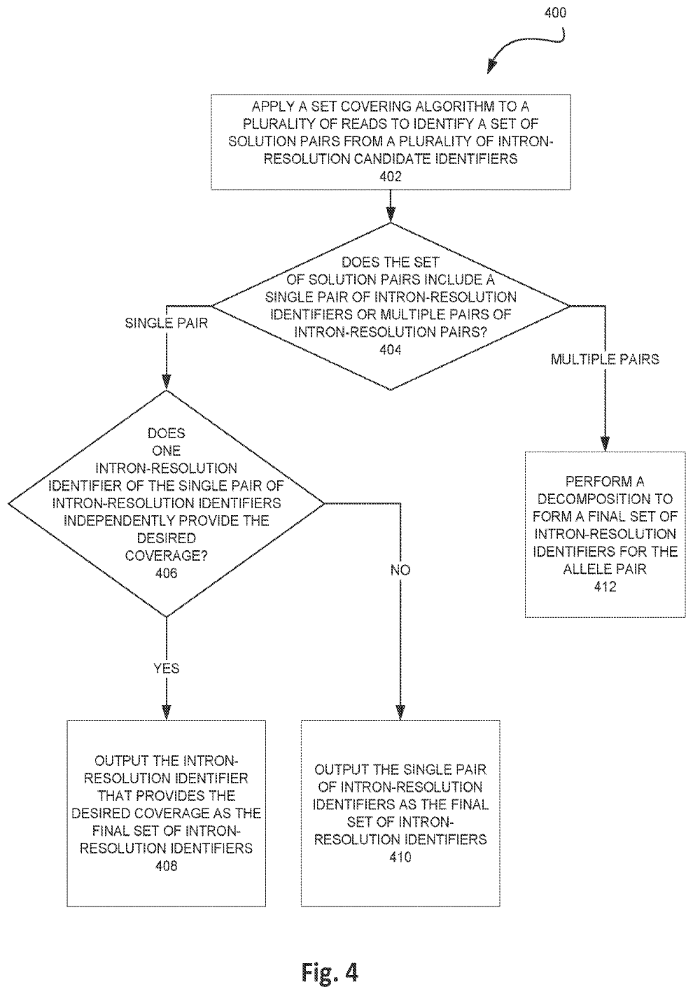

FIG. 4 is a flow diagram illustrating an example of a process 400 for generating a final set of intron-resolution identifiers in accordance with one or more embodiments. Process 400 is one example of a process that is implemented by evaluation system 100 or at least a portion of evaluation system 100 in FIG. 1. Process 400 may be implemented by, for example, allelic type generator 104 in FIG. 1. Process 400 is one example of a process that may be included in step 308 in FIG. 3 described above.

Step 402 includes applying a set covering algorithm to a plurality of reads to identify a set of pairs of intron-resolution identifiers from a plurality of intron-resolution candidate identifiers. The plurality of intron-resolution candidate identifiers may be, for example, the plurality of intron-resolution candidate identifiers identified in step 306 in FIG. 3 described above.

In general, given a universe U of items and a family S of subsets of U, a set cover is a subfamily C⊆S whose union is U. One example of a set covering algorithm finds a set cover C that contains the fewest number of sets. In step 402, the universe U of items includes, for example, reads 116, and S is the family of all known HLA alleles which may be identified in, for example, data store 114 in FIG. 1. Each subset of S, corresponding to a single HLA allele, is defined to be the reads compatible with that allele, where compatibility is determined by sequence similarity of the read to part of the allele. In some embodiments, the set covering algorithm used in step 402 is a modified set covering algorithm in which the HLA alleles are grouped by genes (e.g., HLA-A, HLA-B, HLA-C, etc.). A constrained set cover C*⊆S is one that contains only up to a few (e.g., one, two, etc.) alleles from each gene. The modified set covering algorithm finds the constrained set cover whose union U*⊆U has maximum cardinality N relative to the total cardinality M=|U| over all possible constrained set covers.

Step 402 includes identifying a single pair of intron-resolution identifiers or multiple pairs of intron-resolution identifiers as the set covering solution. In some examples, a pair of intron-resolution identifiers identified in step 402 provides 100% coverage of the plurality of reads such that the maximum number, N, of reads covered is equal to the total number, M, of reads in the plurality of reads. In other examples, a pair of intron-resolution identifiers identified in step 402 provides less than 100% coverage. In this manner, each pair of intron-resolution identifiers identified in step 402 may, for example, explain all or nearly all reads of the plurality of reads. Each pair of intron-resolution identifiers identified in step 402 is a solution pair.

In some embodiments, step 402 includes or process 400 includes, after step 402, generating an alert when N is less than M by some threshold number or percentage (e.g., Nis more than 1%, 2%, 3%, 5%, 10%, etc. less than M).

In some embodiments, step 402 includes computing a “goodness” of the one or more solutions found via the set covering algorithm. This “goodness” metric may be, for example, the fraction of reads that are explained by the various alleles.

Using a set covering algorithm in step 402 helps reduce or eliminate the issues associated with gene cross-talk. Cross-talk occurs when a read aligns to alleles for two different genes. For example, a read that aligns to both an allele for the HLA-A gene and the HLA-B gene is one example of cross-talk. Cross-talk can artificially inflate coverage. For example, cross-talk can artificially inflate the amount of coverage of reads provided by an allele. The set covering algorithm solves collectively for all HLA genes that have pairwise cross-talk.

Step 404 includes determining whether a single pair or multiple pairs of intron-resolution identifiers have been identified. If a single pair was identified, step 406 is performed.

Step 406 includes determining whether either intron-resolution identifier of the single pair of intron-resolution identifiers independently provides the desired coverage. For example, the single pair of intron-resolution identifiers includes a first intron-resolution identifier for a first allele and a second intron-resolution identifier for a second allele. Step 406 includes, for example, determining whether the maximum number, N1, of reads covered by the first intron-resolution identifier is equal to the maximum number, N, of reads covered by the pair of intron-resolution identifiers. In some embodiments, step 406 includes determining whether the maximum number, N1, of reads covered by the first intron-resolution identifier is within a selected range of the maximum number, N, of reads covered by the pair of intron-resolution identifiers. Step 406 includes, for example, determining whether the maximum number, N2, of reads covered by the second intron-resolution identifier is equal to N. In some embodiments, step 406 includes determining whether the maximum number, N2, of reads covered by the second intron-resolution identifier is within the selected range of N. The selected range, discussed above with respect to N1 and N2, may be, for example, one read, two reads, three reads, etc. In step 406, if either N1 or N2 is equal to (or, in some embodiments, within the selected range of N) the corresponding intron-resolution identifier is considered as providing the desired coverage.

If a single intron-resolution identifier of the single pair of intron-resolution identifiers provides the desired coverage, step 408 is performed. Otherwise, step 410 is performed. Step 408 includes outputting the single intron-resolution identifier as the final set of intron-resolution identifiers. Step 410 includes outputting the pair of intron-resolution identifiers as the final set of intron-resolution identifiers.

With reference again to step 404, if multiple pairs of intron-resolution identifiers are identified, step 412 is performed. Step 412 includes performing a decomposition to form the final set of intron-resolution identifiers. The decomposition in step 412 may be performed in various ways. In one or more embodiments, the decomposition includes processing a disjunction, D, of the pairs, (Xᵢ,Xⱼ), where i≠j, and Xᵢ and Xⱼ are members of a set of possible intron-resolution identifiers X. The decomposition algorithm generates a pair (Y,Z) of disjunctions, where Y⊆X and Z⊆X are disjoint and the cross-product of Y and Z is a superset of D. This pair of disjunctions includes, for example, multiple intron-resolution identifiers for at least one allele of the first allele and the second allele. Step 412 may include performing, for example, hierarchical clustering. As used herein, “hierarchical clustering,” also known as hierarchical cluster analysis, includes one or more algorithms that group similar objects into groups called clusters. The endpoint is a set of clusters, where each cluster is distinct from each other cluster, and the objects within each cluster are broadly similar to each other.

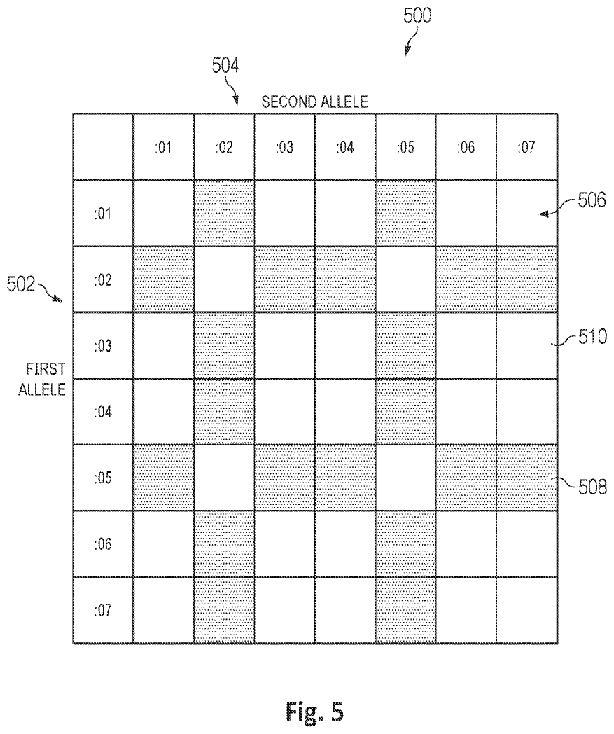

FIG. 5 is an illustration of an example of a matrix representation of a disjunction of pairs of intron-resolution identifiers in accordance with one or more embodiments. Matrix 500 represents, for example, the coverage provided by multiple pairs of intron-resolution identifiers. These multiple pairs of intron-resolution identifiers are a disjunction of solution pairs yielded by the set covering algorithm, such as the set covering algorithm applied in step 402 in FIG. 4 described above. Each solution pair is the pair of alleles that provides the best coverage of the reads (e.g., reads 116 from FIG. 1).

In one or more examples, matrix 500 represents the various intron-resolution identifiers for the two alleles of the solution pairs of intron-resolution identifiers for which the decomposition process in step 412 in FIG. 4 is performed. For example, a total number, S, of solution pairs of intron-resolution identifiers, when viewed collectively, includes x different intron-resolution identifiers for the first allele and y different intron-resolution identifiers for the second allele. Matrix 500 represents these x different intron-resolution identifiers and y different intron-resolution identifiers in an unpaired, decomposed manner.

For example, each pair of intron-resolution identifiers includes an intron-resolution identifier for a first allele and an intron-resolution identifier for a second allele. Matrix 500 includes rows 502 that represent all of the intron-resolution identifiers in the pairs for the first allele. In one or more examples, when the intron-resolution identifiers are 8-digit identifiers, each of rows 502 is represented by the seventh and eighth digits of the corresponding 8-digit identifier. Matrix 500 further includes columns 504 that represent all of the intron-resolution identifiers in the pairs for the second allele. In one or more examples, when the intron-resolution identifiers are 8-digit identifiers, each of columns 504 is represented by the seventh and eighth digits of the corresponding 8-digit identifier.

Elements 506 in matrix 500 indicate whether the pair formed by a corresponding row and a corresponding column is one of the solution pairs. For example, elements 506 include shaded elements 508 and non-shaded elements 510. A shaded element of shaded elements 508 represents, for example, that the pair of intron-resolution identifiers formed by the corresponding row and corresponding column is a solution pair. A non-shaded element of non-shaded elements 510 represents, for example, that the pair of intron-resolution identifiers formed by the corresponding row and corresponding column is not a solution pair.

Decomposition can be performed via, for example, hierarchical clustering to transform the disjunction of pairs of intron-resolution identifiers represented in matrix 500 into a pair of disjunctions. Thus, decomposition unpairs the pairs of intron-resolution identifiers. Hierarchical clustering includes determining similarity or closeness between intron-resolution identifiers. This similarity or closeness may be measured in various ways including, for example, without limitation, Dice distance (or Dice coefficient). Hierarchical clustering is used to identify one or more intron-resolution identifiers for the first allele and one or more intron-resolution identifiers for the second allele.

III.B.2 HLA Typing to Identify Exon-Resolution Identifiers

FIG. 6 is a flow diagram illustrating an example of a process 600 for typing HLA alleles in accordance with one or more embodiments. Process 600 is one example of a process that is implemented by evaluation system 100 or at least a portion of evaluation system 100 in FIG. 1. Process 600 may be implemented by, for example, allelic type generator 104 in FIG. 1. Process 600 is one example of a process that may be included in step 204 in FIG. 2 described above. Process 600 is one example of a process that can be used to generate the input received in step 302 described above.

Step 602 includes receiving a plurality of reads for a sample. The sample is a biological sample. The sample may be, for example, of healthy or normal tissue. The sample may be, for example, of unhealthy or diseased tissue taken at a point in time that is of interest.

Process 600 may optionally include step 603. For example, in one or more embodiments, process 600 includes performing step 603, followed by step 604, as described below. In other embodiments, process 600 includes performing step 604 described below after step 602.

Step 603 includes filtering the plurality of reads to form a filtered plurality of reads. This filtering may be performed in different ways.

In one or more embodiments, the plurality of reads may be aligned against the non-HLA genome, a reference domain of HLA alleles, individual HLA exons, or a combination thereof to identify HLA-related reads. The non-HLA genome may be the portion of the genome excluding HLA genes. The reference domain of HLA alleles may include, for example, all known HLA alleles. An HLA exon is an exon that appears in at least one HLA allele of an HLA gene. The non-HLA genome, reference domain of HLA alleles, HLA exons, or combination thereof may be retrieved from a data store, such as data store 114 in FIG. 1, another data source (e.g., the international IMGT® database), or a combination thereof. Those reads that are HLA-related (e.g., do not align to the non-HLA genome, align to an HLA allele, or align to at least one HLA exon) are considered HLA-related or HLA reads (and may also be referred to as a ‘filtered’ set of reads).

In the same or a different embodiment, the HLA reads are filtered in step 603 by removing any paired-end reads that are duplicates in either the forward or reverse complement direction as such reads may represent undesired artifacts (e.g., PCR duplicate artifacts) to thereby form the filtered plurality of reads. Thus, the filtered plurality of reads formed in step 603 may be filtered via one or two levels of filtering.

Step 604 includes evaluating alignment between at least a portion of the plurality of reads and a series formed by a final set of exon identifiers for each exon position of an HLA gene. Each HLA gene may have a certain number of exons interspersed with introns. Each exon may be considered as having an exon position in the HLA gene. When 604 is performed after step 603, the portion of the plurality of reads used in evaluating alignment is the filtered plurality of reads. When 604 is performed after step 602, the portion of the plurality of reads used in evaluating alignment includes all of the plurality of the reads. Step 604 may include evaluating the alignment after the alignment has already been performed or may include performing the alignment and evaluating the alignment.

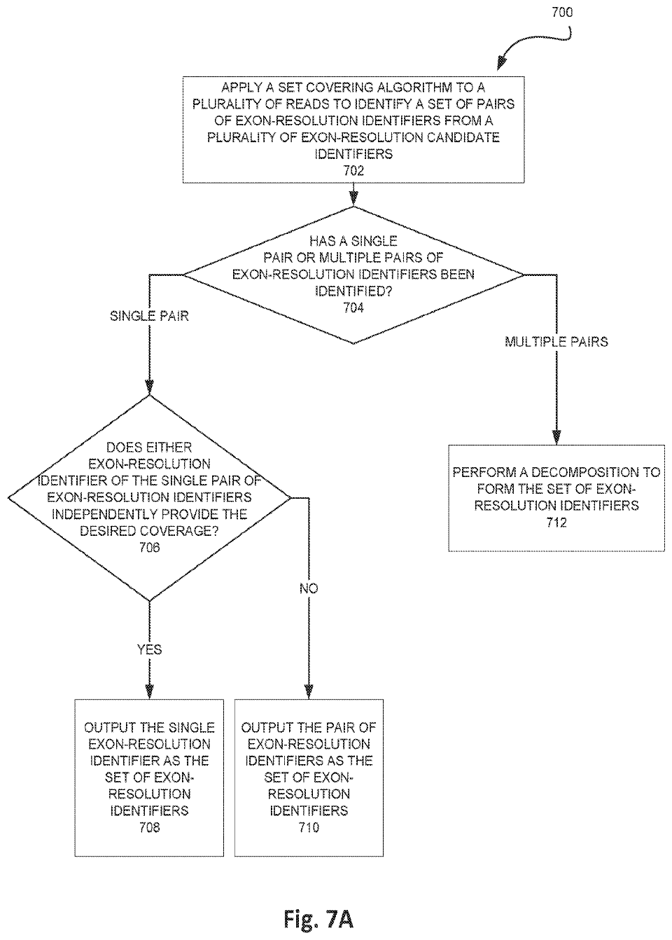

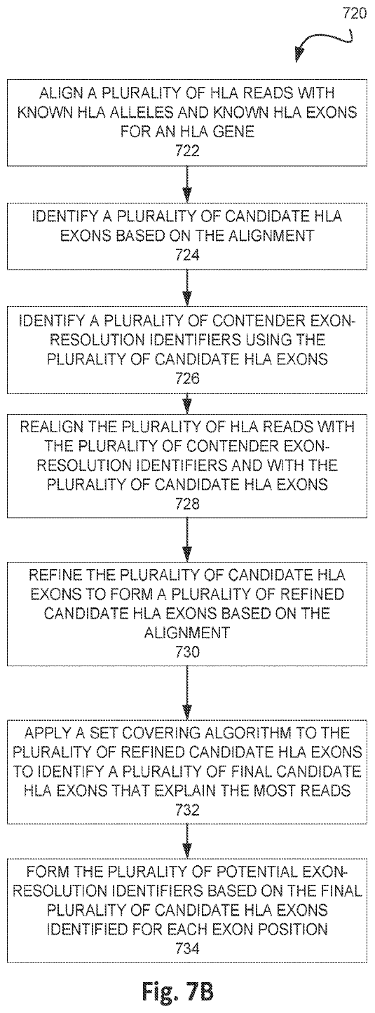

In step 604, the series of exon-resolution identifiers for an HLA gene may be generated based on potential exon-resolution identifiers for a subdomain of the reference domain of HLA alleles that corresponds to the HLA gene. This subdomain includes the portion of HLA alleles within the reference domain of HLA alleles that are possible for the given HLA gene (e.g., all (unconstrained) possible HLA alleles for the given HLA gene). The alignment described in step 604 can be performed using, for example, paired-end alignment. A paired-end alignment is an alignment between an allele and a paired-end read. In other words, an alignment occurs where the allele aligns to both sequences of the paired-end read. In some embodiments, the series is determined using process 720 in FIG. 7B.

Step 606 includes forming a plurality of exon-resolution candidate identifiers from the series formed by the final set of exon identifiers for each exon position of the HLA gene based on exon coverage. In one or more embodiments, step 606 is performed by using the series of exon-resolution identifiers for the HLA gene to constrain the list of potential exon-resolution identifiers for each exon position based on the final set of exon identifiers for that position to form plurality of exon-resolution candidate identifiers. This may be performed progressively by constraining in order of exon position (e.g., in order of exon position having the most diversity (e.g., sequence variants). This constraining of the potential exon-resolution identifiers (e.g., an initial group of exon-resolution identifiers) may be progressively performed for each exon position until exon positions have been completed or until an exon position for which zero potential exon-resolution identifiers were identified is reached.

In some embodiments, step 606 can be performed by, for example, determining whether a plurality of exons associated with a potential exon-resolution identifier is covered by the plurality of reads by at least a coverage threshold. Step 606 can include, for example, adding a potential exon-resolution identifier to the plurality of exon-resolution candidate identifiers if the plurality of exons associated with the potential exon-resolution identifier is covered by the plurality of reads by the coverage threshold. In some examples, the coverage threshold is 100% coverage. In other examples, the coverage threshold is less than 100% coverage. For example, the coverage threshold may be coverage of all but one or two of the plurality of exons. As another example, the coverage threshold may be coverage of at least 90% of each exon of the plurality of exons (e.g., at least 90% of an exon region is explained by the plurality of reads). Thus, the coverage threshold may be set in various ways.

Step 608 includes generating, for each allele of a set of alleles for the HLA gene, a set of exon-resolution identifiers from the plurality of exon-resolution candidate identifiers. If the set of exon-resolution identifiers includes a single exon-resolution identifier, this indicates homozygosity at the exon-resolution level. If the set of exon-resolution identifiers includes two exon-resolution identifiers, one for each of the two HLA alleles expected for the HLA gene, this indicates heterozygosity at the exon-resolution level.

FIG. 7A is a flow diagram illustrating an example of a process 700 for generating a set of exon-resolution identifiers in accordance with one or more embodiments. Process 700 is one example of a process that is implemented by evaluation system 100 or at least a portion of evaluation system 100 in FIG. 1. Process 700 may be implemented by, for example, allelic type generator 104 in FIG. 1. Process 700 is one example of a process that may be included in step 608 in FIG. 6 described above.

Step 702 includes applying a set covering algorithm to a plurality of reads to identify a set of pairs of exon-resolution identifiers from a plurality of exon-resolution candidate identifiers. The plurality of exon-resolution candidate identifiers may be, for example, the plurality of exon-resolution candidate identifiers identified in step 606 in FIG. 6 described above. Step 702 includes identifying a single pair of exon-resolution identifiers or multiple pairs of exon-resolution identifiers as the set covering solution. In some examples, a pair of exon-resolution identifiers identified in step 702 provides 100% coverage of the plurality of reads such that the maximum number, N, of reads covered is equal to the total number, M, of reads in the plurality of reads. In other examples, a pair of exon-resolution identifiers identified in step 702 provides less than 100% coverage. In this manner, each pair of exon-resolution identifiers identified in step 702 may, for example, explain all or nearly all reads of the plurality of reads.

Using a set covering algorithm to identify the set of pairs of exon-resolution identifiers helps reduce or eliminate the issues associated with gene cross-talk. As previously discussed, cross-talk occurs when a read aligns to alleles for two different genes. For example, a read that aligns to both an allele for the HLA-A gene and the HLA-B gene is one example of cross-talk. Cross-talk can artificially inflate exon coverage. For example, cross-talk can artificially inflate the amount of coverage of exon regions of an allele sequence provided by reads.

In some embodiments, step 702 includes computing a “goodness” of the one or more solutions found via the set covering algorithm. This “goodness” metric may be, for example, the fraction of reads that are explained by the various alleles. The set covering algorithm searches for the set of pairs of exon-resolution identifiers that identifies the most reads.

Step 704 includes determining whether a single pair or multiple pairs of exon-resolution identifiers have been identified. If a single pair was identified, step 706 is performed.

Step 706 includes determining whether either exon-resolution identifier of the single pair of exon-resolution identifiers independently provides the desired coverage. For example, the single pair of exon-resolution identifiers includes a first exon-resolution identifier for a first allele and a second exon-resolution identifier for a second allele. Step 706 includes, for example, determining whether the maximum number, N1, of reads covered by the first exon-resolution identifier is equal to or within a selected range of the maximum number, N, of reads covered by the pair of exon-resolution identifiers. Step 706 includes, for example, determining whether the maximum number, N2, of reads covered by the second exon-resolution identifier is equal to or within a selected range of N. The selected range may be, for example, one read, two reads, three reads, etc. In step 706, if either N1 or N2 is equal to or within the selected range of N, the corresponding exon-resolution identifier is considered as providing the desired coverage.

If a single exon-resolution identifier of the single pair of exon-resolution identifiers provides the desired coverage, step 708 is performed. Otherwise, step 710 is performed. Step 708 includes outputting the single exon-resolution identifier as the final set of exon-resolution identifiers for the HLA gene. Step 710 includes outputting the pair of exon-resolution identifiers as the set of exon-resolution identifiers for the HLA gene.

With reference again to step 704, if multiple pairs of exon-resolution identifiers are identified, step 712 is performed. Step 712 includes performing a decomposition to form the set of exon-resolution identifiers. The decomposition in step 712 may be performed in various ways. In one or more embodiments, the decomposition includes identifying, from a disjunction of the pairs of exon-resolution identifiers, a pair of disjunctions. This pair of disjunctions includes, for example, multiple exon-resolution identifiers for at least one allele of the first allele and the second allele. Step 712 may include performing, for example, hierarchical clustering.

FIG. 7B is a flow diagram illustrating an example of a process 720 for identifying a final set of exon identifiers for each exon position of an HLA gene in accordance with one or more embodiments. Process 720 is one example of a process that is implemented by evaluation system 100 or at least a portion of evaluation system 100 in FIG. 1. Process 720 may be implemented by, for example, allelic type generator 104 in FIG. 1. Process 720 is one example of a process that may be used to form the plurality of potential exon-resolution identifiers described in step 604 in FIG. 6 described above.

Step 722 includes aligning a plurality of HLA reads with a subdomain of HLA alleles and HLA exons for an HLA gene. As previously described with respect to step 604 in FIG. 6, this subdomain of HLA alleles includes the portion of HLA alleles within the reference domain of HLA alleles that are possible for the given HLA gene (e.g., all (unconstrained) possible HLA alleles for the given HLA gene). The subdomain of HLA alleles and HLA exons, or both may be retrieved from a data store, such as data store 114 in FIG. 1, another data source (e.g., the international IMGT® database), or a combination thereof. The plurality of HLA reads in step 722 may be, for example, the filtered plurality of reads formed in step 603. The alignment with respect to the subdomain of HLA alleles is performed using exon-resolution identifiers for the subdomain of HLA alleles.

Step 724 includes identifying a set of potential exon identifiers for each exon position of the HLA gene based on the alignment. A potential exon identifier is an exon identifier (e.g., identifier representing the nucleotide sequence of exon) for an HLA exon that is fully covered by at least one HLA read of the plurality of HLA reads. Coverage is determined separately for the 5′ half and 3′ half of each read alignment. For example, the “total coverage level” for a nucleotide position of the nucleotide sequence corresponding to the potential exon identifier is the total count for the number of reads (both 5′ halves and 3′ halves) that match the nucleotide at this nucleotide position when the reads are aligned to the nucleotide sequence corresponding to the potential exon identifier. A “coverage hole” is a nucleotide position of the nucleotide sequence corresponding to the potential exon identifier that is not covered by the 5′ halves or the 3′ halves of any of the HLA reads. In other words, a “coverage hole” is a nucleotide position that has a total coverage level of zero A “coverage anomaly” can occur when (a) there are two adjacent nucleotide positions that have total coverage levels that differ in a statistically significant manner (e.g., by more than a selected threshold, as determined by performing a binomial test, etc.) or (b) the individual 5′ half and 3′ half coverage levels at a given nucleotide position are different in a statistically significant manner (e.g., by more than a selected threshold, as determined by performing a binomial test, etc.).

An exon identifier with a nucleotide sequence for which there exists a coverage hole is excluded from the potential exon identifiers. An exon identifier with a nucleotide sequence for which there exists a coverage anomaly is excluded from the potential exon identifiers.

Step 726 includes identifying an initial group of exon-resolution identifiers using the set of potential exon identifiers identified for each exon position. An exon-resolution identifier in this initial group of exon-resolution identifiers is one in which all exons of the corresponding HLA allele has been identified as one of the potential exon identifiers. For example, an exon-resolution identifier in this initial group of exon-resolution identifiers is one in which the exon at each exon position of the corresponding HLA allele is included in the set of potential exon identifiers determined for that exon position.

Step 728 includes realigning the plurality of HLA reads with the initial group of exon-resolution identifiers and with the sets of potential exon identifiers. In step 728, for each exon position of the HLA gene, each read is allowed to align to every potential exon identifier in the corresponding set of potential exon identifiers identified from step 724. This is a lenient alignment in the sense that it allows an evaluation of mismatches (e.g., up to X mismatches, where X may be tunable) between nucleotides of an HLA read and the nucleotides of the nucleotide sequence corresponding to the potential exon identifier.

For a subject having a homozygous HLA allele, the expectation would be to see one potential exon identifier that is perfectly matched throughout the corresponding nucleotide sequence's length (except for the occasional sequencing error(s) reflected in a mismatch in step 728). For a heterozygous subject having two different alleles, however, no potential exon identifier would be perfectly matched because the reads from the first allele that leniently align to a first potential exon identifier would have mismatches and the reads from the second allele that leniently align to a second potential exon identifier would similarly have mismatches. The pattern of these mismatches would be complementary. For example, the nucleotide positions of the mismatches observed with the first potential exon identifier would be the same nucleotide positions of the mismatches observed with the second potential exon identifier. In this manner, heterozygous exon pairs can be inferred.

Step 730 includes further refining the sets of potential exon identifiers to form a set of candidate exon identifiers for each exon position of the HLA gene based on the alignment. In step 730, the set of potential exon identifiers for each exon position may be refined by excluding any potential exon identifier with a nucleotide sequence for which there exists a coverage hole. The set of candidate exon identifiers for a given exon position may include one or more single candidate exon identifiers (e.g., for a homozygous allele), one or more pairs of candidate exon identifiers (e.g., for a heterozygous allele), or both.

Step 732 includes identifying a series formed by a final set of exon identifiers for each exon position of the HLA gene using a set covering algorithm and the sets of candidate exon identifiers. The set covering algorithm used in step 732 may be similar to the set covering algorithm described above in step 702 with respect to FIG. 7A (in relation to step 608 in FIG. 6), and/or the set covering algorithm described above in step 402 in FIG. 4 (in relation to step 308 in FIG. 3. For example, the set covering algorithm is used to identify the one or more exon identifiers and/or pairs of exon identifiers that explain the most reads for each exon position of the HLA gene. Step 732 constrains the set of candidate exon identifiers for each exon position of the HLA gene to the final set of exon identifiers for each exon position. In one or more embodiments, the analysis may be performed with respect to closures, where a closure is a set of exon positions in multiple HLA genes (e.g., the second exon position in HLA-B gene and the second position in HLA-C gene) that share reads (e.g., explained via cross-talk).

FIG. 8 is a bipartite graph of allele-read alignments in accordance with one or more example embodiments. Graph 800 shows reads 801 at the top of graph 800 and alleles 802 at the bottom of graph 800. In one or more embodiments, alleles 802 are represented in the bipartite graph by their exon-resolution identifiers. Reads 801 are show in sections 804 (e.g., colored sections) that represent different genes. Alignments 806 illustrate the alignments between reads and alleles for the various genes. For example, pair of alleles 808 is identified as a solution pair covering section 810 corresponding to the HLA-B gene. Pair of alleles 812 is identified as a solution pair covering section 814 corresponding to the HLA-C gene. Allele 816 is an example of a single allele covering all reads for section 818 corresponding to the HLA-A gene. Cross-talk alignment 820 is one example of cross-talk in which a particular allele is identified as aligning with reads from two different genes (e.g., HLA-H and HLA-A).

III.C Evaluation of HLA Loss Using Intron-Resolution Identifiers

The various embodiments described herein provide methods and systems for evaluating HLA loss based on intron-resolution identifiers that have been identified for the alleles for an HLA gene. For example, the methods and systems include quantifying alignments between sample reads and the intron-resolution identifiers. These methods and systems further include performing a statistical analysis of this quantification to generate HLA loss information, such as HLA loss information 124 described above with respect to FIG. 1.

III.C.1 Alignment of Set of Alleles with Reads

FIG. 9 is a flow diagram illustrating an example of a process 900 for quantifying allele-read alignments for HLA genes in accordance with one or more embodiments. Process 900 is one example of a process that is implemented by evaluation system 100 or at least a portion of evaluation system 100 in FIG. 1. Process 900 may be implemented by, for example, HLA loss evaluator 110 in FIG. 1. Process 900 may be implemented by, for example, alignment analyzer 106 in FIG. 1. Process 900 is one example of a process that may be included in step 208 in FIG. 2 described above.

Step 902 includes receiving a first set of intron-resolution identifiers for a first allele for an HLA gene and a second set of intron-resolution identifiers for a second allele for the HLA gene. In one or more embodiments, the first allele and the second allele are the same such as the first set of intron-resolution identifiers and the second set of intron-resolution identifiers.

Step 904 includes receiving a first plurality of reads for a first sample and a second plurality of reads for a second sample. The first plurality of reads, the second plurality of reads, or both may be reads generated via, for example, WES or WGS. The first sample may be, for example, a sample of healthy or normal tissue. The second sample may be, for example, a sample of unhealthy or diseased tissue (e.g., a tumor). In other embodiments, the first sample and the second sample are samples of tissue taken at first and second points in time, respectively, in the progression of a disease (e.g., a tumor, cancer, etc.).

Step 906 includes identifying a first allele sequence for the first allele for the HLA gene using a selected one of the first set of intron-resolution identifiers and a second allele sequence for the second allele for the HLA gene using a selected one of the second set of intron-resolution identifiers. In step 906, in some embodiments, multiple combinations of alleles may be made when the first set of intron-resolution identifiers, the second set of intron-resolution identifiers, or both include multiple intron-resolution identifiers. The combination used for step 906 comprising a selected one of the first set of intron-resolution identifiers and/or a selected one of the second set of intron-resolution identifiers may be performed in different ways. For example, without limitation, the selection is performed via random selection. In other embodiments, the selection is performed in an ordered manner (e.g., selecting the first one of the first set of intron-resolution identifiers, selecting the first one of the second set of intron-resolution identifiers alphanumerically, etc.).

Step 908 includes quantifying allele-read alignments between the first allele sequence and each of the first plurality of reads and the second plurality of reads and between the second allele sequence and each of the first plurality of reads and the second plurality of reads to form an alignment output. Step 908 may include, for example, generating an alignment output based on alignment using the first allele sequence, the second allele sequence, the first plurality of reads, and the second plurality of reads. Step 908 includes, for example, but is not limited to, counting the number of alignments (e.g., exact alignments, alignments within 1 or 2 base pairs, alignments within 3 or 4 base pairs, etc.) between different combinations of the first allele and the second allele with the first sample and the second sample. For example, step 908 can include generating a first count for a number of first allele and first sample alignments, a second count for a number of first allele and second sample alignments, a third count for a number of second allele and first sample alignments, and a fourth count for a number of second allele and second sample alignments. The first allele and first sample alignments are alignments between the first allele sequence and the first plurality of reads associated with the first sample. The first allele and second sample alignments are alignments between the first allele sequence and the second plurality of reads associated with the second sample. The second allele and first sample alignments are alignments between the second allele sequence and the first plurality of reads. The second allele and second sample alignments are alignments between the second allele sequence and the second plurality of reads.

In other embodiments, the alignment output in step 908 takes a different form. The alignment output may include, for example, but is not limited to, ratios, percentages, or other types of quantification formats that characterize the allele-read alignments between the first allele sequence, the second allele sequence, the first plurality of reads, and the second plurality of reads. As one example, the alignment output may include a first ratio and a second ratio. The first ratio is a ratio of the number of alignments between the first allele sequence and the first plurality of reads and the number of alignments between the first allele sequence and the second plurality of reads. The second ratio is a ratio of the number of alignments between the second allele sequence and the first plurality of reads and the number of alignments between the second allele sequence and the second plurality of reads.

In various embodiments, steps 906 and 908 are repeated for various combinations of selected ones from the first set of intron-resolution identifiers and selected ones of the second set of intron-resolution identifiers. For example, all possible combinations using each possible intron-resolution identifier for the first allele and each possible intron-resolution identifier for the second allele may be evaluated to generate alignment output.

FIG. 10 illustrates allele-read alignments and alignment counts in accordance with one or more embodiments. The alignment information includes allele-read alignments 1000 and quadrant 1002. Allele-read alignments 1000 include alignments between a first allele sequence identified by a first intron-resolution identifier (e.g., a first allele) and reads from a first (e.g., normal) sample, between the first allele sequence and reads from a second (e.g., tumor) sample, between a second allele sequence identified by a second intron-resolution identifier (e.g., a second allele) and reads from the first sample, and between the second allele sequence and reads from the second sample.

Quadrant 1002 thus identifies the corresponding counts of these four different types of alignments. Specifically, quadrant 1002 identifies a first count 1004 for the number of first allele and first sample alignments, a second count 1006 for the number of first allele and second sample alignments, a third count 1008 for the number of second allele and first sample alignments, and a fourth count 1010 for the number of second allele and second sample alignments.

III.C.2 Evaluation of HLA Loss Based on Allele-Read Alignments

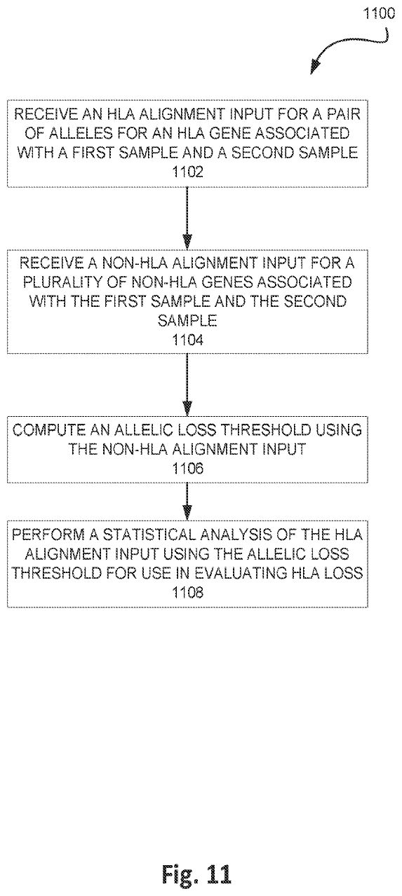

FIG. 11 is a flow diagram illustrating an example of a process 1100 for analyzing HLA allele expression in accordance with one or more embodiments. Process 1100 is one example of a process that is implemented by evaluation system 100 or at least a portion of evaluation system 100 in FIG. 1. Process 1100 may be implemented by, for example, HLA loss evaluator 110 in FIG. 1. Process 1100 may be implemented by, for example, statistics generator 108 in FIG. 1. Process 1100 is one example of a process that may be included in step 210 in FIG. 2 described above.

Step 1102 includes receiving an HLA alignment input for a pair of alleles for an HLA gene associated with a first sample and a second sample. The HLA alignment input includes information about alignment between reads from the first sample and the second sample and HLA alleles. The HLA alignment input includes, for example, the alignment output generated in step 908 in FIG. 9. In some examples, the HLA alignment input includes the alignment output generated via multiple iterations of step 908 (e.g., iterations as described above for various combinations of selected ones of the first set of intron-resolution identifiers and selected ones of the second set of intron-resolution identifiers).

Step 1104 includes receiving a non-HLA alignment input for a plurality of non-HLA genes associated with the first sample and the second sample. This non-HLA alignment input is used to provide training data for a statistical model. The statistical model is, in one or more examples, a beta-binomial model. The non-HLA alignment input provides information regarding the gene (e.g., allelic) read count variation between the first sample and the second sample.

Step 1106 includes computing an allelic loss threshold using the non-HLA alignment input. Step 1106 includes, for example, computing an allelic loss threshold based on the gene variation described by or otherwise inferred from the non-HLA alignment input.

Step 1108 includes performing a statistical analysis of the HLA alignment input using the allelic loss threshold for use in evaluating HLA loss. The statistical analysis performed in step 1108 may be implemented in various ways. In one or more embodiments, the statistical analysis includes computing t-statistics using the beta-binomial model described above. In other embodiments, the statistical analysis is performed using Z-scores, analysis of variance (ANOVA), one or more other statistical algorithms or techniques, or a combination thereof. The results or information generated by the statistical analysis in step 1108 may be referred to as a statistical output.

Although process 900 in FIG. 9 and process 1100 in FIG. 11 are described separately, in some embodiments, process 900 and process 1100 are combined or otherwise integrated as a single process. Further, process 1100 may be repeated for different allele pairs.

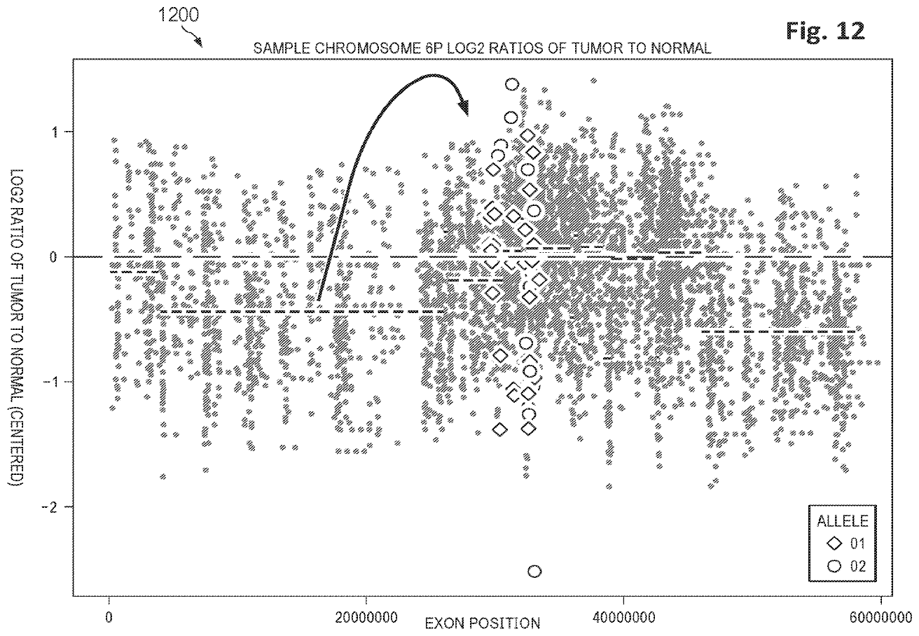

FIG. 12 illustrates an example of a plot of gene variation between two samples in accordance with one or more embodiments. Plot 1200 identifies gene variation between two samples, a normal sample and a tumor sample using log 2 ratios of the alignment counts for the two samples. Plot 1200 identifies gene variation for non-HLA genes and HLA genes (e.g., two HLA alleles). Plot 1200 is one example of a type of statistical output that may be generated via, for example, step 1108 in FIG. 11. In some examples, plot 1200 is one example of a type of output that is generated using a statistical output generated via step 1108 in FIG. 11.

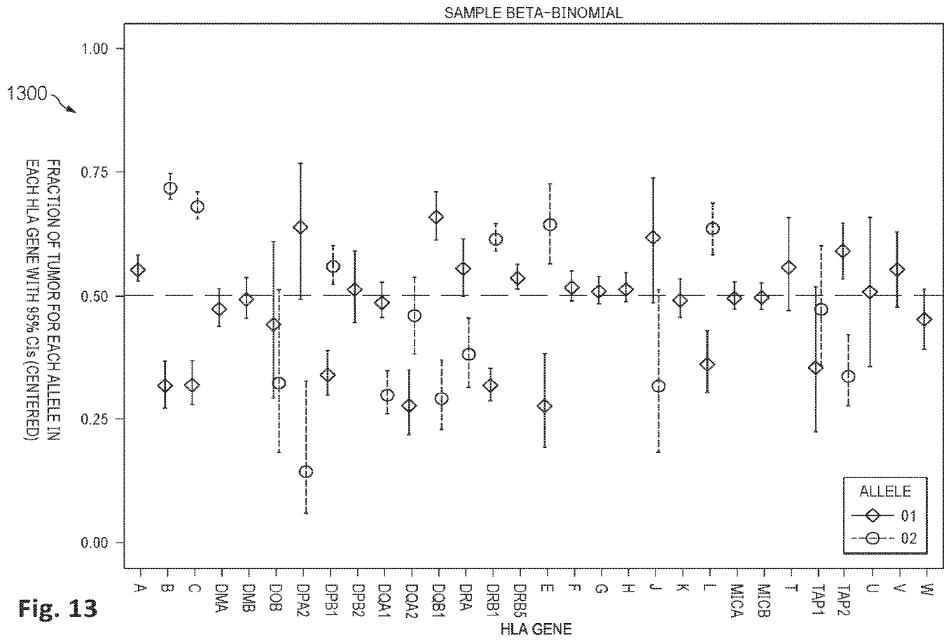

FIG. 13 illustrates an example of a plot generated using a beta-binomial model in accordance with one or more embodiments. Plot 1300 identifies 95% confidence intervals dual to the t-statistics for the various HLA alleles of the HLA genes in a subject. Plot 1300 may be generated using, for example, the information provided in plot 1200 in FIG. 12. Plot 1300 is one example of a type of statistical output that may be generated via, for example, step 1108 in FIG. 11. In some examples, plot 1300 is one example of a type of output that is generated using a statistical output generated via step 1108 in FIG. 11.

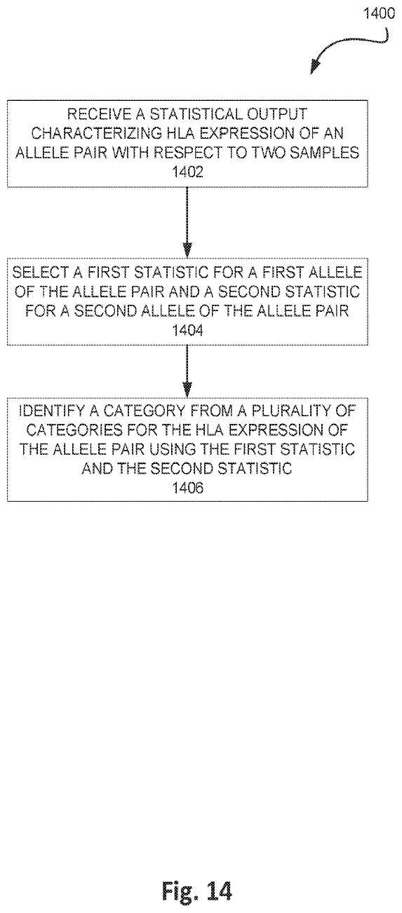

FIG. 14 is a flow diagram illustrating an example of a process 1400 for evaluating HLA loss in accordance with one or more embodiments. Process 1400 is one example of a process that is implemented by evaluation system 100 or at least a portion of evaluation system 100 in FIG. 1. Process 1400 may be implemented by, for example, HLA loss evaluator 100 in FIG. 1. In some examples, process 1400 is implemented by, for example, statistics generator 148 in FIG. 1. Process 1400 is one example of a process that may be included in step 212 in FIG. 2 described above.

Step 1402 includes receiving a statistical output characterizing HLA expression of an allele pair with respect to two samples. Step 1402 may include, for example, receiving a statistical output generated via step 1108 in FIG. 10 described above.

Step 1404 includes selecting a first statistic for a first allele of the allele pair and a second statistic for a second allele of the allele pair. Step 1404 includes, for example, identifying a t-statistic for the first allele and a t-statistic for the second allele.

Step 1406 includes identifying a category from a plurality of categories for the HLA expression of the allele pair using the first statistic and the second statistic. The plurality of expression categories may include, for example, but are not limited to, normal expression, single deletion, double deletion, LOH (e.g., copy-neural LOH), single amplification, and double amplification. Step 1406 includes, for example, plotting an intersection of the t-statistic for the first allele and the t-statistic for the second allele and comparing this intersection to regions or ideal intersections corresponding to the plurality of categories on a Voronoi diagram. The cutoffs or boundaries of the various regions in the Voronoi diagram are customizable to provide higher or lower confidence levels as needed. Further, the cutoffs or boundaries of the various regions in the Voronoi diagram may be customizable to reflect an amount of stromal (e.g., non-tumor) content in a sample (e.g., a sample of tumor tissue).

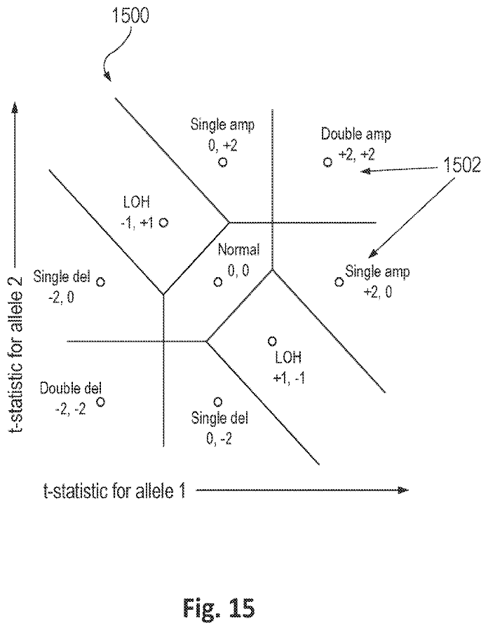

FIG. 15 illustrates an example of a Voronoi diagram in accordance with one or more embodiments. Voronoi diagram 1500 includes regions 1502 to which intersections between the t-statistics of two alleles of an allele pair for an HLA gene can be mapped. Each of regions 1502 identifies a corresponding category that characterizes an expression of the allele pair. Regions 1502 of Voronoi diagram represent one example of a plurality of categories for HLA expression such as, for example, the plurality of categories described in step 1406 in FIG. 14.

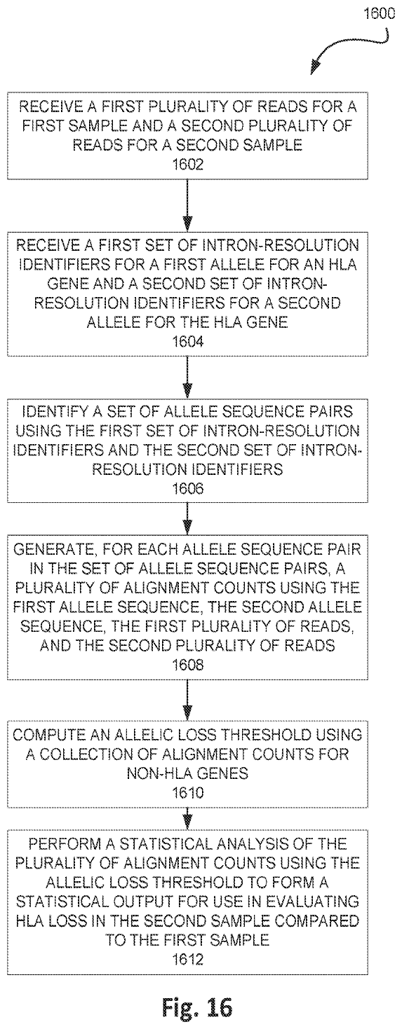

FIG. 16 is a flow diagram illustrating an example of evaluating HLA loss in accordance with one or more embodiments. Process 1600 is one example of a process that is implemented by evaluation system 100 or at least a portion of evaluation system 100 in FIG. 1. Process 1600 may be implemented by, for example, HLA loss evaluator 110 in FIG. 1. Process 1600 is one example of a process that may be used to perform step 210 and step 212 in FIG. 2 described above.

Step 1602 includes receiving a first plurality of reads for a first sample and a second plurality of reads for a second sample. The first sample and the second sample may be a normal sample and a tumor sample, respectively. In other examples, the first sample and the second sample may be samples of a tumor taken at two different points in time.

Step 1604 includes receiving a first set of intron-resolution identifiers for a first allele for an HLA gene and a second set of intron-resolution identifiers for a second allele for the HLA gene. In some examples, the first set of intron-resolution identifiers includes a single intron-resolution identifier that is identical to a single intron-resolution identifier of the second set of intron-resolution identifiers, indicating homozygosity.

Step 1606 includes identifying a set of allele sequence pairs using the first set of intron-resolution identifiers and the second set of intron-resolution identifiers. Each allele sequence pair in the set of allele sequence pairs includes a first allele sequence for the first allele and a second allele sequence for the second allele.

Step 1608 includes generating, for each allele sequence pair in the set of allele sequence pairs, a plurality of alignment counts using the first allele sequence, the second allele sequence, the first plurality of reads, and the second plurality of reads.

Step 1610 includes computing an allelic loss threshold using a collection of alignment counts for non-HLA genes. For example, alignment between the non-HLA genes and both the first plurality of reads and the second plurality of reads may be used to determine a baseline gene expression variation. The alignment counts for these non-HLA genes may be used to train a model, such as, for example, a beta-binomial model. This beta-binomial model may be used to compute the allelic loss threshold in step 1610.

Step 1612 includes performing a statistical analysis of the plurality of alignment counts using the allelic loss threshold to form a statistical output for use in evaluating HLA loss in the second sample compared to the first sample. Step 1612 may include, for example, computing t-statistics for the first allele and the second allele. These t-statistics can be used to estimate an occurrence of HLA loss.

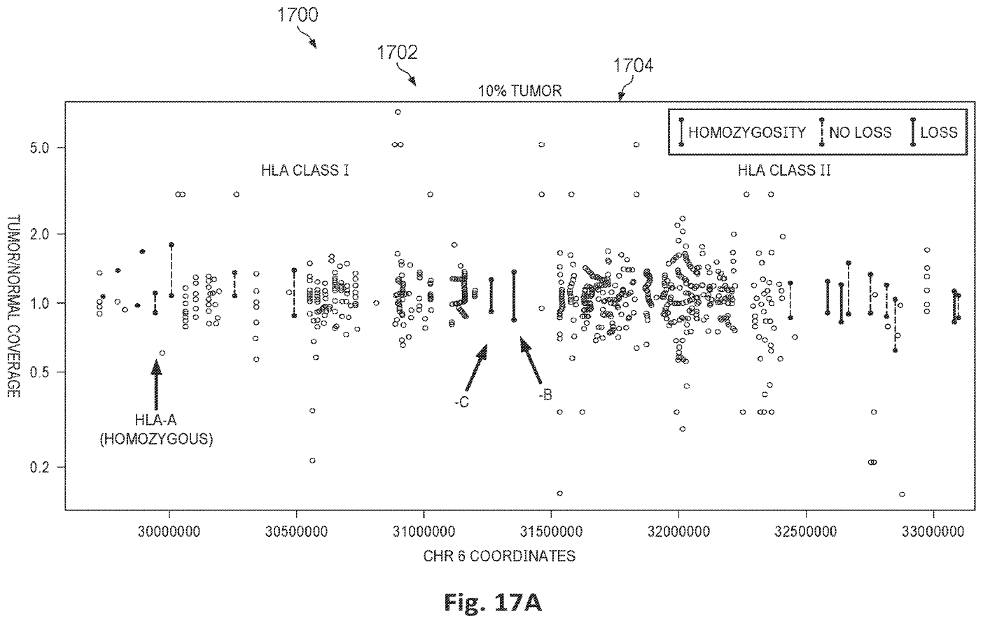

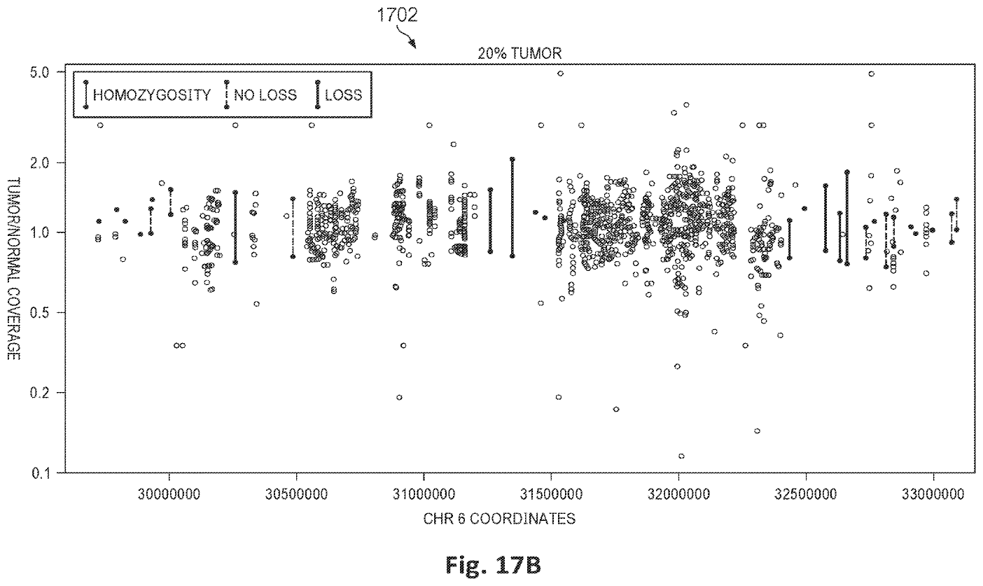

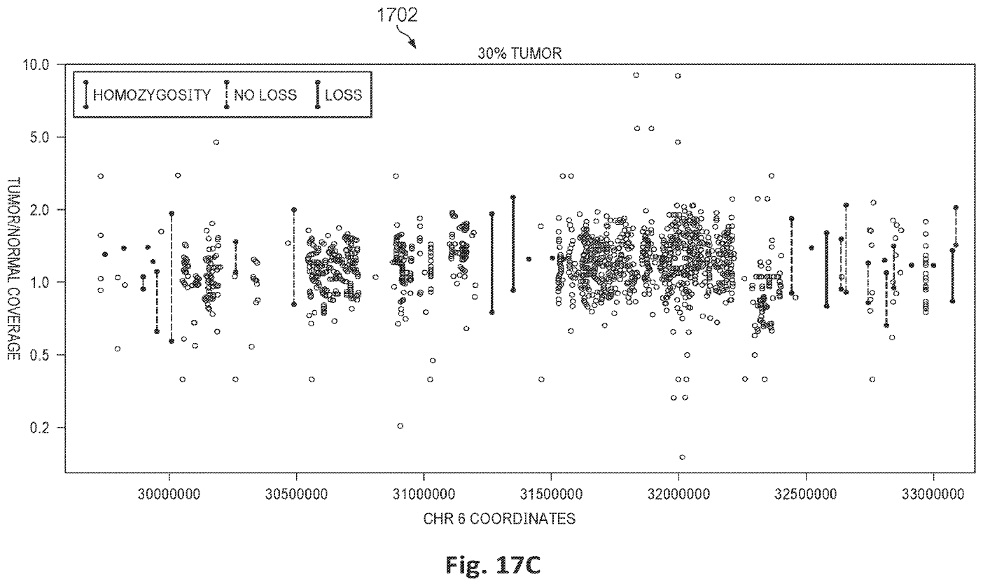

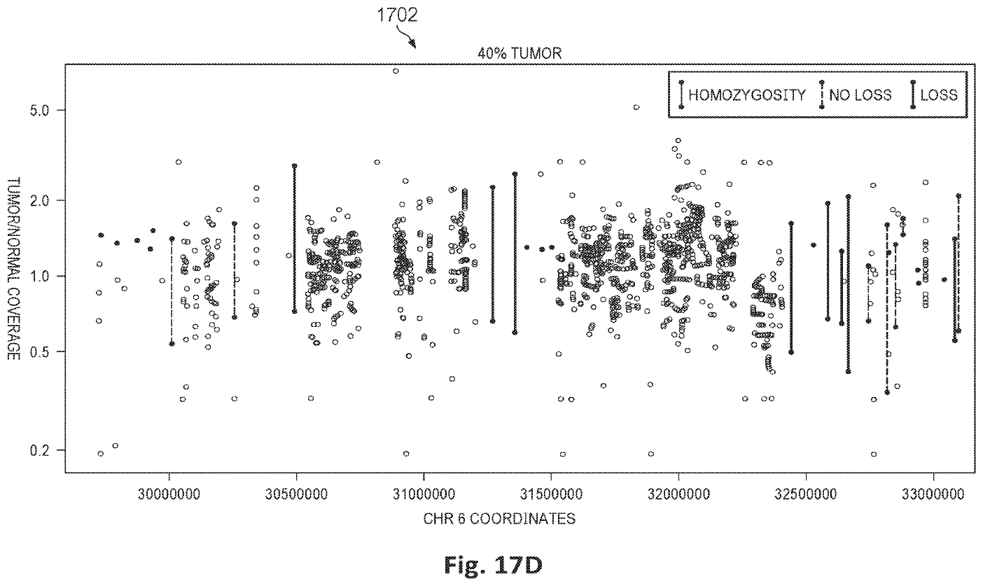

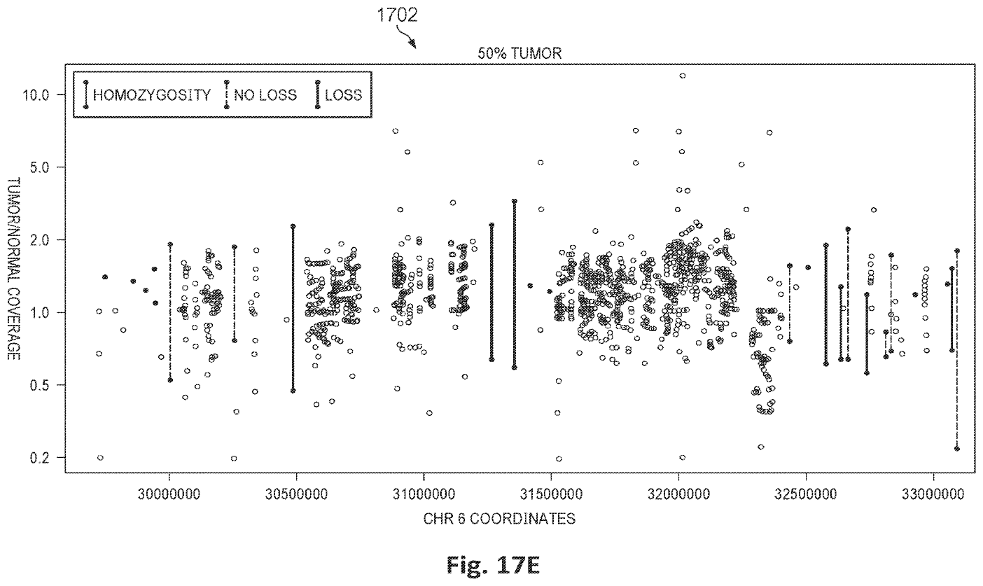

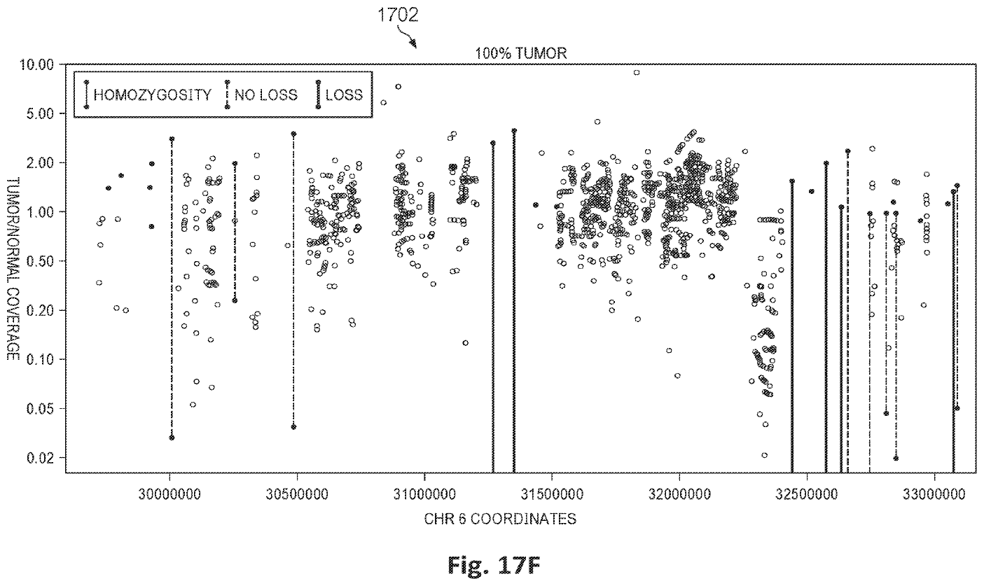

FIGS. 17A-17F illustrate a plot series showing HLA loss detection in various tumor samples in accordance with one or more embodiments. Plot series 1700 in FIGS. 17A-17F includes a group of plots 1702, each of which illustrates the detection of HLA loss for a corresponding tumor sample. This detection of HLA loss is performed based on the methods described by the various embodiments herein. In some embodiments, this detection of HLA loss is performed using process 1600 in FIG. 16. Plot series 1700 may be one example of a type of statistical output generated via step 1612 in FIG. 16. Group of plots 1702 includes plot 1704, which shows that detection of HLA loss is possible even in tumor samples in which the percentage of tumor cells in that tumor sample is as low as 10%.

III.D Decision-Making Based on Evaluation of HLA Loss

The information provided by the processes described above (e.g., process 1100 in FIG. 11, process 1400 in FIG. 14, process 1600 in FIG. 16) can be used to make various types of decisions with respect to at least one of treating or predicting the progression or outcome of a disease such as a tumor or cancer. In one or more embodiments, these processes provide a way of detecting (e.g., estimating) HLA loss in one sample as compared to another sample.

Once HLA loss (e.g., loss of one or more HLA alleles) has been detected, this information can be used to develop and/or personalize immunotherapy, including T cell therapy. For example, T cell cancer therapy can be personalized to account for the loss of certain HLA alleles that would prevent, for example, T cells from reacting to a neoantigen associated with those HLA alleles. Thus, it would be important to develop a T cell therapy that can be activated in a subject based on HLA alleles for which loss of expression (e.g., via absence of expression or reduced expression) has not been detected.

For example, peptides may be presented to the cell surface of a cell with one or more of a subject's HLA alleles (e.g., HLA-A, HLA-B. HLA-C) for immune surveillance. If an antigen (e.g., peptide) appears foreign to the immune system, that cell is killed by the immune system. If HLA allele loss has been detected, a prediction can be made regarding which foreign antigens would have been presented by the lost HLA allele. This type of prediction would help refine the selection of foreign antigens used as targets for tumor therapy (e.g., tumor vaccines). In some cases, if a subject has lost many or all HLA alleles, a determination may be made that the subject is not a good candidate for a tumor vaccine and should be considered for other types of therapies (e.g., therapies involving NK cells).

Neoantigen vaccines can prime a subject's T cells to recognize and attack cancer cells expressing one or more particular tumor neoantigens. This approach can generate a tumor-specific immune response that spares healthy cells while targeting tumor cells. The individualized vaccine may be engineered or selected based on the information generated by the various embodiments described above.

An immunotherapy such as, for example, without limitation, a cancer treatment may include collecting a sample (e.g., a blood sample) from a subject. T cells can be isolated and stimulated. The isolation can be performed using, for example, density gradient sedimentation (e.g., and centrifugation), immunomagnetic selection, and/or antibody-complex filtering. The stimulation may include, for example, antigen-independent stimulation, which may use a mitogen (e.g., PHA or Con A) or anti-CD3 antibodies (e.g., to bind to CD3 and activate the T-cell receptor complex) and anti-CD28 antibodies (e.g., to bind to CD28 and stimulate T cells). A set of peptides (e.g., mutant peptides) can be selected to use in the treatment of the subject based on the information provided by the various embodiments described above corresponding to predictions as to whether and/or an extent to which each of the set of peptides would bind to an MHC molecule (or HLA molecule) of the subject, be presented by the MHC molecule of the subject and/or trigger an immune response in the subject. For example, the set of peptides can be selected based on the detection of HLA loss within one or more tumor samples. This HLA loss may include the loss of one or more HLA alleles.

In some embodiments, the set of peptides (or precursors thereof) can be used to produce mutant peptide (for example, neoantigen) specific T cells. For example, peripheral blood T cells can be isolated from a subject and contacted with one or more mutant peptides to induce mutant peptide-specific T-cells populations that can be administered to a subject. In some examples, the T cell receptor sequence of the mutant peptide-reactive T cells can be sequenced. Once the T-cell receptor sequence (e.g., amino-acid T-cell receptor sequence) is obtained, T cells can be engineered to include the T cell receptor that specifically recognizes the mutant peptide. These engineered T cells can then be administered to a subject. See, e.g., Matsuda et al. “Induction of Neoantigen-Specific Cytotoxic T Cells and Construction of T-cell Receptor Engineered T Cells for Ovarian Cancer,” Clin. Cancer Res. 1-11 (2018), hereby incorporated by reference in its entirety for all purposes. The T cells can be expanded in vitro and/or ex vivo prior to administration to a subject. The subject may then be administered (e.g., infused with) a composition that includes the expanded population of T cells. In one or more embodiments, the treatment is administered to an individual in an amount effective to, for example, prime, activate and expand T cells in vivo.

Thus, the above examples provide some examples of different types of immunotherapies that may be developed based on HLA loss detection. The detection of HLA loss can be used to personalize immunotherapy (e.g., personalize a cancer immunotherapy), determine when to include and when to exclude an antigen that would be presented by an HLA allele as a potential target for an immunotherapy, and/or inform other decisions regarding immunotherapy.

### IV. Computer-Implemented System

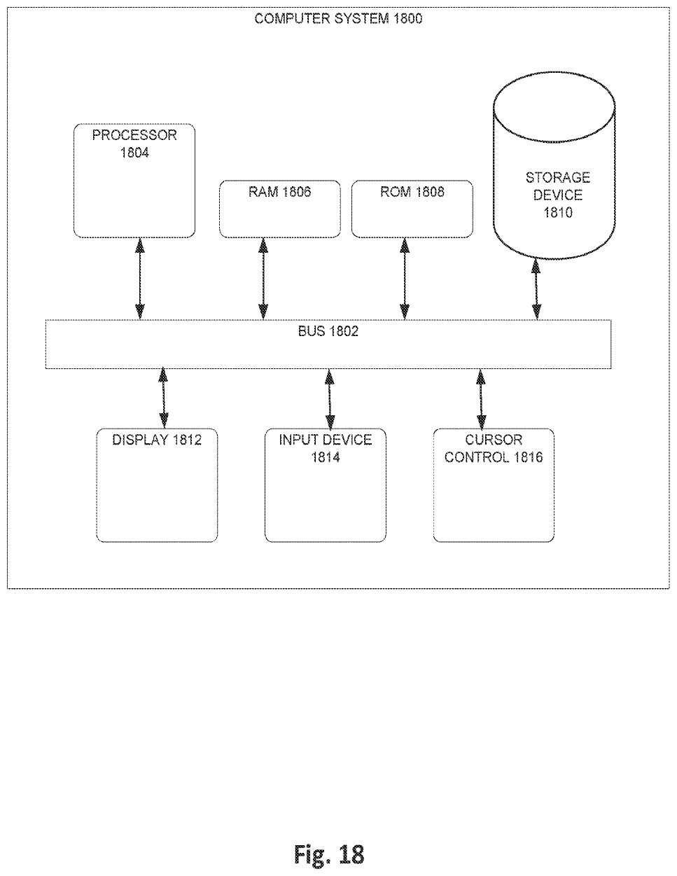

FIG. 18 is a block diagram illustrating an example of a computer system in accordance with various embodiments. Computer system 1800 may be an example of one implementation for computer system 102 described above in FIG. 1. In one or more examples, computer system 1800 can include a bus 1802 or other communication mechanism for communicating information, and a processor 1804 coupled with bus 1802 for processing information. In various embodiments, computer system 1800 can also include a memory, which can be a random-access memory (RAM) 1806 or other dynamic storage device, coupled to bus 1802 for determining instructions to be executed by processor 1804. Memory also can be used for storing temporary variables or other intermediate information during execution of instructions to be executed by processor 1804. In various embodiments, computer system 1800 can further include a read-only memory (ROM) 1808 or other static storage device coupled to bus 1802 for storing static information and instructions for processor 1804. A storage device 1810, such as a magnetic disk or optical disk, can be provided and coupled to bus 1802 for storing information and instructions.

In various embodiments, computer system 1800 can be coupled via bus 1802 to a display 1812, such as a cathode ray tube (CRT) or liquid crystal display (LCD), for displaying information to a computer user. An input device 1814, including alphanumeric and other keys, can be coupled to bus 1802 for communicating information and command selections to processor 1804. Another type of user input device is a cursor control 1816, such as a mouse, a joystick, a trackball, a gesture-input device, a gaze-based input device, or cursor direction keys for communicating direction information and command selections to processor 1804 and for controlling cursor movement on display 1812. This input device 1814 typically has two degrees of freedom in two axes, a first axis (e.g., x) and a second axis (e.g., y), that allows the device to specify positions in a plane. However, it should be understood that input devices 1814 allowing for three-dimensional (e.g., x, y and z) cursor movement are also contemplated herein.

Consistent with certain implementations of the present teachings, results can be provided by computer system 1800 in response to processor 1804 executing one or more sequences of one or more instructions contained in RAM 1806. Such instructions can be read into RAM 1806 from another computer-readable medium or computer-readable storage medium, such as storage device 1810. Execution of the sequences of instructions contained in RAM 1806 can cause processor 1804 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 1804 for execution. Such a medium can take many forms, including but not limited to, non-volatile media, volatile media, and transmission media. Examples of non-volatile media can include, but are not limited to, optical, solid state, magnetic disks, such as storage device 1810. Examples of volatile media can include, but are not limited to, dynamic memory, such as RAM 1806. Examples of transmission media can include, but are not limited to, coaxial cables, copper wire, and fiber optics, including the wires that comprise bus 1802.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1804 of computer system 1800 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 1800 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1800, whereby processor 1804 would execute the analyses and determinations provided by these engines, subject to instructions provided by any one of, or a combination of, the memory components RAM 1806, ROM, 1808, or storage device 1810 and user input provided via input device 1814.

### V. Recitation of Embodiments

Embodiment 1. A method for typing major histocompatibility complex (MHC) alleles, the method comprising: receiving a set of exon-resolution identifiers for an allele pair associated with an MHC gene, wherein an exon-resolution identifier of the set of exon-resolution identifiers for a corresponding allele of the allele pair describes an allele group, a specific allele protein, and exon region information for the corresponding allele; receiving a plurality of reads for a sample; identifying, for each exon-resolution identifier of the set of exon-resolution identifiers, a set of intron-resolution identifiers to form a plurality of intron-resolution candidate identifiers, wherein an intron-resolution identifier of the set of intron-resolution identifiers for the corresponding allele describes the allele group, the specific allele protein, the exon region information, and intron region information for the corresponding allele; and generating a final set of intron-resolution identifiers for the allele pair from the plurality of intron-resolution candidate identifiers.

Embodiment 2. The method of embodiment 1, wherein the set of exon-resolution identifiers including a single exon-resolution identifier indicates that the allele pair is homozygous at an exon level of resolution.

Embodiment 3. The method of embodiment 1, wherein the set of exon-resolution identifiers including at least two exon-resolution identifiers indicates that the allele pair is heterozygous at an exon level of resolution.

Embodiment 4. The method of any one of embodiments 1-3, wherein generating the final set of intron-resolution identifiers comprises: generating the final set of intron-resolution identifiers for the allele pair from the plurality of intron-resolution candidate identifiers using the plurality of reads and a set covering algorithm.

Embodiment 5. The method of any one of embodiments 1-4, wherein generating the final set of intron-resolution identifiers comprises: applying a set covering algorithm to the plurality of reads to identify a set of solution pairs from the plurality of intron-resolution candidate identifiers, each solution pair of the set of solution pairs including two different intron-resolution candidate identifiers from the plurality of intron-resolution candidate identifiers.

Embodiment 6. The method of embodiment 5, wherein generating the final set of intron-resolution identifiers further comprises: performing, in response to the set of solution pairs including multiple solution pairs, a decomposition of the multiple solution pairs to form the final set of intron-resolution identifiers for the allele pair.

Embodiment 7. The method of embodiment 6, wherein the performing comprises: decomposing the multiple solution pairs using hierarchical clustering to form the final set of intron-resolution identifiers for the allele pair.

Embodiment 8. The method of embodiment 5, wherein the generating comprises: determining that the set of solutions pairs includes a single pair of intron-resolution identifiers; and outputting a selected intron-resolution identifier of the single pair of intron-resolution identifiers as the final set of intron-resolution identifiers when the selected intron-resolution identifier provides a same amount of coverage of the plurality of reads as the single pair of intron-resolution identifiers.

Embodiment 9. The method of any one of embodiments 1-8, further comprising: generating the set of exon-resolution identifiers from a plurality of exon-resolution candidate identifiers using a set covering algorithm.

Embodiment 10. The method of embodiment 9, further comprising: evaluating alignment between at least a portion of the plurality of reads and a plurality of potential exon-resolution identifiers for an MHC gene; and forming the plurality of exon-resolution candidate identifiers from the plurality of potential exon-resolution identifiers based on exon coverage.

Embodiment 11. The method of embodiment 10, further comprising: filtering the plurality of reads to form a filtered plurality of reads, wherein the alignment is evaluated between the filtered plurality of reads and the plurality of potential exon-resolution identifiers.

Embodiment 12. The method of embodiment 10 or embodiment 11, wherein forming the plurality of exon-resolution candidate identifiers comprises: adding a potential exon-resolution identifier of the plurality of potential exon-resolution identifiers to the plurality of exon-resolution candidate identifiers if a plurality of exons associated with the potential exon-resolution identifier is covered by the plurality of reads by a coverage threshold.

Embodiment 13. The method of any one of embodiments 1-12, further comprising: applying a set covering algorithm to the plurality of reads to identify a set of solution pairs from a plurality of exon-resolution candidate identifiers, each solution pair of the set of solution pairs including two different exon-resolution candidate identifiers from the plurality of exon-resolution candidate identifiers.

Embodiment 14. The method of embodiment 13, further comprising: performing, in response to the set of solution pairs including multiple solution pairs, a decomposition of the multiple solution pairs to form the set of exon-resolution identifiers for the allele pair.

Embodiment 15. The method of embodiment 14, wherein the performing comprises: decomposing the multiple solution pairs using hierarchical clustering to form the set of exon-resolution identifiers for the allele pair.

Embodiment 16. The method of embodiment 13, further comprising: determining that the set of solutions pairs includes a single pair of exon-resolution identifiers; and outputting a selected exon-resolution identifier of the single pair of exon-resolution identifiers as the set of exon-resolution identifiers when the selected exon-resolution identifier provides a same amount of coverage of the plurality of reads as the single pair of exon-resolution identifiers.

Embodiment 17. The method of any one of embodiments 10-16, further comprising: identifying the plurality of potential exon-resolution identifiers for the MHC gene by constraining the plurality of potential exon-resolution identifiers to a plurality of final candidate exons.

Embodiment 18. The method of any one of embodiments 10-17, further comprising: identifying the plurality of final candidate exons using a set covering algorithm.

Embodiment 19. The method of any one of embodiments 10-18, further comprising: identifying a plurality of candidate MHC exons, wherein each of the plurality of candidate MHC exons is fully covered by at least one read of the at least a portion of the plurality of reads; and identifying the plurality of final candidate exons from the plurality of candidate MHC exons.

Embodiment 20. The method of any one of embodiments 1-19, wherein: the sample is selected as one of a group consisting of a sample of healthy tissue and a sample of unhealthy tissue; and the plurality of reads is generated via at least one of whole-exome sequencing (WES) or whole genome sequencing (WGS).

Embodiment 21. The method of any one of embodiments 1-20, further comprising: generating a first set of intron-resolution identifiers for a first allele of the allele pair for the MHC gene and a second set of intron-resolution identifiers for a second allele of the allele pair using the final set of intron-resolution identifiers, wherein the each of the first set of intron-resolution identifiers and the second set of intron-resolution identifiers includes a same, single intron-resolution identifier when the allele pair is homozygous.

Embodiment 22. The method of any one of embodiments 1-21, further comprising: evaluating MHC loss between the sample and another sample using the final set of intron-resolution identifiers.

Embodiment 23. The method of any one of embodiments 1-22, wherein the plurality of reads includes a plurality of paired-end reads.

Embodiment 24. The method of any one of embodiments 1-23, wherein the MHC gene is a human leukocyte antigen (HLA) gene and wherein the MHC alleles are HLA alleles.

Embodiment 25. The method of any one of embodiments 1-24, wherein the final set of intron-resolution identifiers is generated based on a series formed by a final set of exon identifiers for each exon position of the MHC gene.

Embodiment The method of any one of embodiments 1-25, wherein the set of exon-resolution identifiers is generated based on a series formed by a final set of exon identifiers for each exon position of the MHC gene.

Embodiment The method of any one of embodiments 1-24, wherein the final set of intron-resolution identifiers is generated based on three set covering algorithms used at an exon identifier level, an exon-resolution identifier level, and an intron-resolution identifier level.

Embodiment The method of any one of embodiments 1-24, wherein the final set of intron-resolution identifiers is generated based on a three-tier refinement at an exon identifier level, an exon-resolution identifier level, and an intron-resolution identifier level.

Embodiment 29. A method for comparing reads with allele sequences for major histocompatibility complex (MHC) genes for use in evaluating MHC allelic expression, the method comprising: receiving a first set of intron-resolution identifiers for a first allele for an MHC gene and a second set of intron-resolution identifiers for a second allele for the MHC gene; receiving a first plurality of reads for a first sample and a second plurality of reads for a second sample; and identifying a first allele sequence for the first allele for the MHC gene using a selected one of the first set of intron-resolution identifiers and a second allele sequence for the second allele for the MHC gene using a selected one of the second set of intron-resolution identifiers; and quantifying allele-read alignments between the first allele sequence and each of the first plurality of reads and the second plurality of reads and between the second allele sequence and each of the first plurality of reads and the second plurality of reads to form an alignment output.

Embodiment 30. The method of embodiment 29, wherein quantifying the allele-read alignments comprises: quantifying the allele-read alignments with increased accuracy based on using the first allele sequence and the second allele sequence corresponding to the selected one of the first set of intron-resolution identifiers and the selected one of the second set of intron-resolution identifiers.

Embodiment 31. The method of embodiment 29, wherein quantifying the allele-read alignments comprises: counting a number of first allele and first sample alignments between the first allele sequence and the first plurality of reads; and counting a number of first allele and second sample alignments between the first allele sequence and the second plurality of reads.

Embodiment 32. The method of embodiment 31, further comprising: evaluating MHC loss associated with the first allele using the first number of alignments and the second number of alignments.

Embodiment 33. The method of embodiment 29, wherein quantifying the allele-read alignments comprises: counting a number of second allele and first sample alignments between the second allele sequence and the first plurality of reads; and counting a number of second allele and second sample alignments between the second allele sequence and the second plurality of reads.

Embodiment 34. The method of embodiment 33, further comprising: evaluating MHC loss associated with the second allele using the first number of alignments and the second number of alignments.

Embodiment 35. The method of any one of embodiments 29-34, further comprising: performing a statistical analysis using the alignment output to generate a statistical output for use in evaluating MHC loss.

Embodiment 36. The method of any one of embodiments 29-35, wherein the first set of intron-resolution identifiers is identical to the second set of intron-resolution identifiers when the first allele and the second allele are homozygous.

Embodiment 37. The method of any one of embodiments 29-36, wherein the plurality of reads is generated via at least one of whole-exome sequencing (WES) or whole genome sequencing (WGS).

Embodiment 38. The method of any one of embodiments 29-37, wherein the first sample is a sample of healthy tissue and the second sample is a sample of unhealthy tissue.

Embodiment 39. The method of any one of embodiments 29-38, wherein each intron-resolution identifier of the first set of intron-resolution identifiers and of the second set of intron-resolution identifiers is an 8-digit MHC allele identifier.

Embodiment 40. The method of any one of embodiments 29-39, further comprising: evaluating MHC loss using the alignment output.

Embodiment 41. The method of any one of embodiments 29-40, wherein the MHC gene is a human leukocyte antigen (HLA) gene.

Embodiment 42. A method for analyzing major histocompatibility complex (MHC) allelic loss between samples, the method comprising: receiving an MHC alignment input for a pair of alleles for an MHC gene associated with a first sample and a second sample; receiving a non-MHC alignment input for a plurality of non-MHC genes associated with the first sample and the second sample; computing an allelic loss threshold using the non-MHC alignment input; and performing a statistical analysis of the MHC alignment input using the allelic loss threshold to form a statistical output for use in evaluating MHC loss.

Embodiment 43. The method of embodiment 42, further comprising: evaluating loss of at least one allele of the pair of alleles using the statistical output.

Embodiment 44. The method of any one of embodiments 42 and 43, wherein receiving the MHC alignment input comprises: receiving a first number of alignments corresponding to a first allele of the pair of alleles and the first sample; and receiving a second number of alignments corresponding to the first allele of the pair of alleles and the second sample.

Embodiment 45. The method of embodiment 44, wherein receiving the MHC alignment input further comprises: receiving a third number of alignments corresponding to a second allele of the pair of alleles and the first sample; and receiving a fourth number of alignments corresponding to the second allele of the pair of alleles and the second sample.

Embodiment 46. The method of any one of embodiments 42-45, wherein performing the statistical analysis comprises: computing a first t-statistic for a first allele of the pair of alleles; and computing a second t-statistic for a second allele of the pair of alleles.

Embodiment 47. The method of embodiment 46, further comprising: analyzing the first t-statistic and the second t-statistic to categorize expression of the pair of alleles.

Embodiment 48. The method of embodiment 47, wherein analyzing the first t-statistic and the second t-statistic comprises: analyzing the first t-statistic and the second t-statistic using a Voronoi diagram.

Embodiment 49. The method of any one of embodiments 42-48, further comprising: categorizing an expression of the pair of alleles as one of a plurality of categories using the statistical output, wherein the plurality of categories includes normal expression, single deletion, double deletion, loss of heterozygosity, single amplification, and double amplification.

Embodiment 50. The method of any one of embodiments 42-49, wherein performing the statistical analysis comprises: computing Z-scores for the pair of alleles.

Embodiment 51. The method of any one of embodiments 42-50, wherein performing the statistical analysis comprises: performing the statistical analysis using analysis of variance (ANOVA).

Embodiment 52. The method of any one of embodiments 42-51, further comprising: generating a report that contains at least a portion of the statistical output, wherein the report includes, for an allele of the MHC gene, at least one of a confidence interval for a log-ratio of tumor-to-normal copy number for the allele, a t-statistic for the allele, a Z-score for the allele, or a p-value for that allele.

Embodiment 53. The method of any one of embodiments 42-52, further comprising: detecting loss of at least one MHC allele using the statistical output; and personalizing an immunotherapy based on the loss of the at least one MHC allele.

Embodiment 54. The method of any one of embodiments 42-52, further comprising: detecting loss of at least one MHC allele using the statistical output; and determining to exclude a set of antigens that would be presented by the at least one MHC allele from a set of target antigens for an immunotherapy.

Embodiment 55. The method of any one of embodiments 42-52 and 54, further comprising: determining to include an antigen that would be presented by an MHC allele of the pair of alleles on a surface of a tumor cell as a target for an immunotherapy based on the statistical output.

Embodiment 56. The method of any one of embodiments 42-52, further comprising: determining to exclude an antigen that would be presented by an MHC allele of the pair of alleles on a surface of a tumor cell as a target for an immunotherapy based on the statistical output.

Embodiment 57. The method of any one of embodiments 53-56, wherein the immunotherapy is selected from a group consisting of a T cell therapy, a personalized cancer therapy, an antigen-specific immunotherapy, an antigen-dependent immunotherapy, a vaccine, and a natural killer (NK) cell therapy.

Embodiment 58. The method of any one of embodiments 42-57, wherein the MHC gene is a human leukocyte antigen (HLA) gene.

Embodiment 59. A method for analyzing major histocompatibility complex (MHC) allelic loss between samples, the method comprising: receiving a first plurality of reads for a first sample and a second plurality of reads for a second sample; and receiving a first set of intron-resolution identifiers for a first allele for an MHC gene and a second set of intron-resolution identifiers for a second allele for the MHC gene; identifying a set of allele sequence pairs using the first set of intron-resolution identifiers and the second set of intron-resolution identifiers, wherein each allele sequence pair in the set of allele sequence pairs includes a first allele sequence for the first allele and a second allele sequence for the second allele; generating, for each allele sequence pair in the set of allele sequence pairs, a plurality of alignment counts using the first allele sequence, the second allele sequence, the first plurality of reads, and the second plurality of reads; computing an allelic loss threshold using a collection of alignment counts for non-MHC genes; and performing a statistical analysis of the plurality of alignment counts using the allelic loss threshold to form a statistical output for use in evaluating MHC loss in the second sample compared to the first sample.

Embodiment 60. The method of embodiment 59, wherein generating, for each allele sequence pair of allele sequences in the set of allele sequence pairs, the plurality of alignment counts comprises: counting a number of first allele and first sample alignments between the first allele sequence of a corresponding allele sequence pair of the set of allele sequence pairs and the first plurality of reads; and counting a number of first allele and second sample alignments between the first allele sequence of the corresponding allele sequence pair and the second plurality of reads.

Embodiment 61. The method of any one of embodiments 59 and 60, wherein generating, for each allele sequence pair of allele sequences in the set of allele sequence pairs, the plurality of alignment counts comprises: counting a number of second allele and first sample alignments between the second allele sequence of a corresponding allele sequence pair of the set of allele sequence pairs and the first plurality of reads; and counting a number of second allele and second sample alignments between the second allele sequence of the corresponding allele sequence pair and the second plurality of reads.

Embodiment 62. The method of any one of embodiments 59-61, wherein performing the statistical analysis comprises: computing a first t-statistic for the first allele having the first allele sequence; and computing a second t-statistic for the second allele having the second allele sequence.

Embodiment 63. The method of any one of embodiments 59-62, further comprising: categorizing an expression of the first allele and the second allele as one of a plurality of categories using the statistical output, wherein the plurality of categories includes normal expression, single deletion, double deletion, loss of heterozygosity, single amplification, and double amplification.

Embodiment 64. The method of any one of embodiments 59-63, wherein the MHC gene is a human leukocyte antigen (HLA) gene and wherein the non-MHC genes are non-HLA genes.

Embodiment 65. A method for evaluating major histocompatibility complex (MHC) copy number alteration, the method comprising: receiving a first plurality of reads for a first sample and a second plurality of reads for a second sample; identifying a final set of intron-resolution identifiers for an allele pair for an MHC gene using the first plurality of reads and a set covering algorithm; identifying a set of allele sequence pairs using the final set of intron-resolution identifiers, wherein each allele sequence pair in the set of allele sequence pairs includes a first allele sequence and a second allele sequence for the allele pair; generating, for each allele sequence pair in the set of allele sequence pairs, a plurality of alignment counts using the first allele sequence, the second allele sequence, the first plurality of reads, and the second plurality of reads; performing a statistical analysis of the plurality of alignment counts to form a statistical output; estimating MHC copy number alteration in the second sample as compared to the first sample using the statistical output.

Embodiment 66. The method of embodiment 65, further comprising: categorizing an expression of the first allele and the second allele as one of a plurality of categories using the statistical output, wherein the plurality of categories includes normal expression, single deletion, double deletion, loss of heterozygosity, single amplification, and double amplification.

Embodiment 67. The method of any one of embodiments 65 and 66, further comprising: detecting loss of at least one of allele of the allele pair using the statistical output; and determining to exclude a set of antigens that would be presented by the at least one allele of the allele pair from a set of target antigens for an immunotherapy.

Embodiment 68. The method of any one of embodiments 65-67, further comprising: determining to include an antigen that would be presented by at least one allele of the allele pair on a surface of a tumor cell as a target for an immunotherapy based on the statistical output.

Embodiment 69. The method of any one of embodiments 65-68, further comprising: determining to exclude an antigen that would be presented by an MHC allele of the pair of alleles on a surface of a tumor cell as a target for an immunotherapy based on the statistical output.

Embodiment 70. The method of any one of embodiments 65 and 66, further comprising: detecting loss of at least one allele of the allele pair using the statistical output; and personalizing an immunotherapy based on the loss of the at least one allele.

Embodiment 71. The method of any one of embodiments 65-70, wherein the immunotherapy is selected from a group consisting of a T cell therapy, a personalized cancer therapy, an antigen-specific immunotherapy, an antigen-dependent immunotherapy, a vaccine, and a natural killer (NK) cell therapy.

Embodiment 72. The method of any one of embodiments 65-71, further comprising: generating a report that contains at least a portion of the statistical output, wherein the report includes, for an allele of the MHC gene, at least one of a confidence interval for a log-ratio of tumor-to-normal copy number for the allele, a t-statistic for the allele, a Z-score for the allele, or a p-value for that allele.

Embodiment 73. The method of any one of embodiments 65-72, wherein the MHC gene is a human leukocyte antigen (HLA) gene.

Embodiment 74. A system comprising: one or more data processors; and a non-transitory computer readable storage medium containing instructions which, when executed on the one or more data processors, cause the one or more data processors to perform part or all of one or more methods disclosed in embodiments 1-73.

Embodiment 75. A computer-program product tangibly embodied in a non-transitory machine-readable storage medium, including instructions configured to cause one or more data processors to perform part or all of one or more methods disclosed in embodiments 1-73.

Embodiment 76. A method comprising one or more methods disclosed in embodiments 1-73.

### VI. Additional Considerations

The headers and subheaders between sections and subsections of this document are included solely for the purpose of improving readability and do not imply that features cannot be combined across sections and subsection. Accordingly, sections and subsections do not describe separate embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements (e.g., elements in block or schematic diagrams, elements in flow diagrams, etc.) without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A method for typing major histocompatibility complex (MHC) alleles, the method comprising:
receiving a set of exon-resolution identifiers for an allele pair associated with an MHC gene,
wherein an exon-resolution identifier of the set of exon-resolution identifiers for a corresponding allele of the allele pair describes an allele group, a specific allele protein, and exon region information for the corresponding allele;
identifying, for each exon-resolution identifier of the set of exon-resolution identifiers, a set of intron-resolution identifiers to form a plurality of intron-resolution candidate identifiers,
wherein an intron-resolution identifier of the set of intron-resolution identifiers for the corresponding allele describes the allele group, the specific allele protein, the exon region information, and intron region information for the corresponding allele; and
generating a final set of intron-resolution identifiers for the allele pair from the plurality of intron-resolution candidate identifiers.

2. The method of claim 1, wherein the set of exon-resolution identifiers including a single exon-resolution identifier indicates that the allele pair is homozygous or heterozygous at an exon level of resolution.

3. (canceled)

4. The method of claim 1, wherein generating the final set of intron-resolution identifiers comprises:
generating the final set of intron-resolution identifiers for the allele pair from the plurality of intron-resolution candidate identifiers using a plurality of reads for a sample and a set covering algorithm.

5. The method of claim 1, wherein generating the final set of intron-resolution identifiers comprises:
applying a set covering algorithm to a plurality of reads for a sample to identify a set of solution pairs from the plurality of intron-resolution candidate identifiers, each solution pair of the set of solution pairs including two different intron-resolution candidate identifiers from the plurality of intron-resolution candidate identifiers.

6. The method of claim 5, wherein generating the final set of intron-resolution identifiers further comprises:
performing, in response to the set of solution pairs including multiple solution pairs, a decomposition of the multiple solution pairs to form the final set of intron-resolution identifiers for the allele pair, wherein the decomposition comprises decomposing the multiple solution pairs using hierarchical clustering to form the final set of intron-resolution identifiers for the allele pair.

7. (canceled)

8. The method of claim 5, wherein the generating comprises:
determining that the set of solution pairs includes a single pair of intron-resolution identifiers; and
outputting a selected intron-resolution identifier of the single pair of intron-resolution identifiers as the final set of intron-resolution identifiers when the selected intron-resolution identifier provides a same amount of coverage of the plurality of reads as the single pair of intron-resolution identifiers.

9. The method of claim 1, further comprising:
generating the set of exon-resolution identifiers from a plurality of exon-resolution candidate identifiers using a set covering algorithm;
evaluating alignment between at least a portion of a plurality of reads for a sample and a plurality of series formed by a final set of exon identifiers for each position of an MHC gene; and
forming the plurality of exon-resolution candidate identifiers from the plurality of series based on exon coverage.

10. (canceled)

11. The method of claim 9, further comprising:
filtering the plurality of reads to form a filtered plurality of reads, wherein the alignment is evaluated between the filtered plurality of reads and the plurality of series formed by the final set of exon identifiers for each exon position of the MHC gene.

12. The method of claim 9, wherein forming the plurality of exon-resolution candidate identifiers comprises:
adding a potential exon-resolution identifier of a plurality of potential exon-resolution identifiers to the plurality of exon-resolution candidate identifiers if a plurality of exons associated with the potential exon-resolution identifier is covered by the plurality of reads by a coverage threshold.

13. The method of claim 1, further comprising:
applying a set covering algorithm to a plurality of reads for a sample to identify a set of solution pairs from a plurality of exon-resolution candidate identifiers, each solution pair of the set of solution pairs including two different exon-resolution candidate identifiers from the plurality of exon-resolution candidate identifiers.

14. The method of claim 13, further comprising:
performing, in response to the set of solution pairs including multiple solution pairs, a decomposition of the multiple solution pairs to form the set of exon-resolution identifiers for the allele pair, wherein the decomposition comprises decomposing the multiple solution pairs using hierarchical clustering to form the set of exon-resolution identifiers for the allele pair.

15. (canceled)

16. The method of claim 13, further comprising:
determining that the set of solution pairs includes a single pair of exon-resolution identifiers; and
outputting a selected exon-resolution identifier of the single pair of exon-resolution identifiers as the set of exon-resolution identifiers when the selected exon-resolution identifier provides a same amount of coverage of the plurality of reads as the single pair of exon-resolution identifiers.

17. The method of claim 9, further comprising:
identifying the plurality of series for the MHC gene by constraining the plurality of series to a plurality of final candidate exons, wherein the plurality of final candidate exons are identified using a set covering algorithm;
identifying a plurality of candidate MHC exons, wherein each of the plurality of candidate MHC exons is fully covered by at least one read of the at least a portion of the plurality of reads; and
identifying the plurality of final candidate exons from the plurality of candidate MHC exons.

18. (canceled)

19. (canceled)

20. The method of claim 1, further comprising:
receiving a plurality of reads for a sample, wherein:
the sample is selected as one of a group consisting of a sample of healthy tissue and a sample of unhealthy tissue; and
the plurality of reads is generated via at least one of whole-exome sequencing (WES) or whole genome sequencing (WGS).

21. The method of claim 1, further comprising:
generating a first set of intron-resolution identifiers for a first allele of the allele pair for the MHC gene and a second set of intron-resolution identifiers for a second allele of the allele pair using the final set of intron-resolution identifiers,
wherein the each of the first set of intron-resolution identifiers and the second set of intron-resolution identifiers includes a same, single intron-resolution identifier when the allele pair is homozygous.

22. The method of claim 1, further comprising:
receiving a plurality of reads for a sample; and
evaluating MHC loss between the sample and another sample using the final set of intron-resolution identifiers, wherein the plurality of reads includes a plurality of paired-end reads, the MHC gene is a human leukocyte antigen (HLA) gene, and the MHC alleles are HLA alleles.

23. (canceled)

24. (canceled)

25. The method of claim 1, wherein the final set of intron-resolution identifiers is generated based on at least one of:
a series formed by a final set of exon identifiers for each exon position of the MHC gene,
three set covering algorithms used at an exon identifier level, an exon-resolution identifier level, and an intron-resolution identifier level, or
a three-tier refinement at an exon identifier level, an exon-resolution identifier level, and an intron-resolution identifier level.

26. The method of claim 1, wherein the set of exon-resolution identifiers is generated based on a series formed by a final set of exon identifiers for each exon position of the MHC gene.

27-73. (canceled)

74. A system comprising:
one or more data processors; and
a non-transitory computer readable storage medium containing instructions which, when executed on the one or more data processors, cause the one or more data processors to:
receive a set of exon-resolution identifiers for an allele pair associated with an MHC gene,
wherein an exon-resolution identifier of the set of exon-resolution identifiers for a corresponding allele of the allele pair describes an allele group, a specific allele protein, and exon region information for the corresponding allele;
identify, for each exon-resolution identifier of the set of exon-resolution identifiers, a set of intron-resolution identifiers to form a plurality of intron-resolution candidate identifiers,
wherein an intron-resolution identifier of the set of intron-resolution identifiers for the corresponding allele describes the allele group, the specific allele protein, the exon region information, and intron region information for the corresponding allele; and
generate a final set of intron-resolution identifiers for the allele pair from the plurality of intron-resolution candidate identifiers.

75. A computer-program product tangibly embodied in a non-transitory machine-readable storage medium, including instructions configured to cause one or more data processors to perform a method comprising:
receiving a set of exon-resolution identifiers for an allele pair associated with an MHC gene,
wherein an exon-resolution identifier of the set of exon-resolution identifiers for a corresponding allele of the allele pair describes an allele group, a specific allele protein, and exon region information for the corresponding allele;
identifying, for each exon-resolution identifier of the set of exon-resolution identifiers, a set of intron-resolution identifiers to form a plurality of intron-resolution candidate identifiers,
wherein an intron-resolution identifier of the set of intron-resolution identifiers for the corresponding allele describes the allele group, the specific allele protein, the exon region information, and intron region information for the corresponding allele; and
generating a final set of intron-resolution identifiers for the allele pair from the plurality of intron-resolution candidate identifiers.
